(19) United States
(12) Patent Application Publication
(10) Pub. No.: US 2015/0186609 A1
(43) Pub. Date: Jul. 2, 2015

(54) DATA CAPABLE STRAPBAND FOR SLEEP MONITORING, COACHING, AND AVOIDANCE

(71) Applicant: Max Everett Utter II, San Francisco, CA (US)

(72) Inventor: Max Everett Utter II, San Francisco, CA (US)

(73) Assignee: AliphCom, San Francisco, CA (US)

(21) Appl. No.: 14/194,495

(22) Filed: Feb. 28, 2014

Related U.S. Application Data
(63) Continuation of application No. 14/145,856, filed on Dec. 31, 2013.

(51) Int. Cl. G06F 19/00; A61B 5/0205; A61B 5/16; A61B 5/11; A61B 5/0488; G01L 1/00; A61B 5/00

(52) U.S. Cl. CPC G06F 19/3406; G01L 1/00; G06F 19/3487; A61B 5/02055; A61B 5/6843; A61B 5/1112; A61B 5/4809; A61B 5/721; A61B 5/7282; A61B 5/4812; A61B 5/742; A61B 5/0022; A61B 5/681; A61B 5/0488; A61B 5/165; A61B 5/4866; A61B 5/02405

(57) ABSTRACT

A wireless data capable strapband operative to detect inflammation, true resting heart rate (TRHR), and fatigue of a user may include sensors for generating motion signals in response to body motion (e.g., accelerometry), for generating force signals in response to force exerted by a body portion, for generating biometric signals indicative of biometric activity in the body (e.g., heart rate, respiration, arousal), and for generating location signals based on user location. The sensor signals may be processed to monitor parameters that may positively or negatively impact a state of sleep of the user (e.g., time of sleep, quality of sleep, hydration, inflammation, contraction, fatigue, TRHR, accelerometry, arousal of the SNS, etc.). Data from the processed signals may be presented (e.g., visually, sound, email, text message, tactile, webpage, etc.) to the user in a format (e.g., reports notifications, coaching, avoidance) intended to instruct/encourage the user to improve their state of sleep.

### CROSS-REFERENCE TO RELATED APPLICATIONS

This Application is a Continuation of Co-Pending U.S. patent application Ser. No. 14/145,856, filed on Dec. 31, 2013, having Attorney Docket No. ALI-282, and titled “Wearable Device To Detect Inflammation”. This Application is also related to the following applications: U.S. patent application Ser. No. 14/073,550, filed on Nov. 6, 2013, having Attorney Docket No. ALI-280, and titled “Protective Covering For Wearable Devices”; U.S. patent application Ser. No. 13/830,860, filed on Mar. 14, 2013, having Attorney Docket No. ALI-152, and titled “Platform For Providing Wellness Assessments And Recommendations Using Sensor Data”; U.S. patent application Ser. No. 13/967,317, filed on Aug. 14, 2013, having Attorney Docket No. ALI-260, and titled “Real-Time Psychological Characteristic Detection Based On Reflected Components Of Light”; and U.S. patent application Ser. No. 13/890,1433, filed on May 8, 2013, having Attorney Docket No. ALI-262, and titled “System And Method For Monitoring The Health Of A User”, all of which are hereby incorporated by reference in their entirety for all purposes.

### FIELD

The present application relates generally to portable electronics, wearable electronics, biometric sensors, personal biometric monitoring systems, location sensing, and more specifically to systems, electronics, structures and methods for wearable devices for inflammation monitoring, contraction monitoring, reporting and health coaching and avoidance for a user.

### BACKGROUND

Systemic inflammation may be a medial indicator, leading indicator or cause of many disease states and ill health known (e.g., genetic disease, communicable disease, the common cold, flu, organ disease, injury, trauma, cancer, etc.). Examples of bodily events that may cause bodily tissues to swell (e.g., capillary dilation, interstitial fluid buildup, etc.) and manifest as inflammation include prolonged stress and sleep deprivation. In the case of sleep deprivation, people often associated a feeling or appearance of puffiness which typically manifests itself around the eyes as bags under the eyes or puffiness of the soft tissues that surround the eyes. Another example is acute inflammation, such as resulting from dosing high levels of sugar due to an insulin response causing blood sugar to be stored in cells and fluid storage in the cells that lead to inflammation. An immune response state of the body (e.g., at the onset of disease or a prolonged disease state) is yet another example of a causation of inflammation. Chronic inflammation may be caused by a bodily system(s) constantly fighting off some prolonged state such as injury to muscles, ligaments, organs and other tissues, and organ disease, for example. Causation of inflammation may be disconnected from or distant the portion(s) of the body where the inflammation manifests itself, as in the case of athletes that over train or injury or organ disease, for example. Systemic inflammation is not typically a localized event and some body event that is the cause of the inflammation will typically be manifested in other areas of the body, such as the fingers of the hands (e.g., any of the digits of the hand including the thumb), or the toes of the foot, for example. In some examples, body tissues may shrink due to lack of hydration and that shrinkage may be most prominently evident in cases of severe dehydration where slight shrinkages of body tissues may be ascertainable through measurement. One or more biometric indicators may be associated with systemic inflammation and/or dehydration and be measured using a variety of biometric or other types of sensors and systems, such as metrics from electrodermal activity of the skin (e.g., galvanic skin response—GSR, etc.), heart rate monitors, body weight, bioimpedance, body temperature, just to name a few. For example, an increase in skin resistance due to dehydration may be detected using a GSR sensor, and EMG sensor, or other sensors for detecting electrodermal activity.

Moreover, environmental, dietary, social conditions and other externalities may impact bodily functions that cause inflammation, such as stress from work, school, relationships, family members, finances, commuting, etc. Systemic inflammation may be used as a biomarker that may be helpful in diagnosing disease or as an indicator of early onset of disease. Ideally, it would be desirable to accurately distinguish between systemic inflammation caused by some bodily event and/or externalities that effect the body as opposed to swelling of body tissues that may be due to added muscle mass (e.g., cell growth leading to muscle tissue growth) or shrinkage of body tissues due to dehydration.

Accordingly, there is a need for a user wearable device that monitors a plurality of bodily metrics and external data to measure inflammation, indicate inflammation to the user, and optionally provide coaching and/or remediation to the user.

### BRIEF DESCRIPTION OF THE DRAWINGS

Various embodiments or examples (“examples”) of the present application are disclosed in the following detailed description and the accompanying drawings. The drawings are not necessarily to scale:

FIGS. 1A-1B depict a cross-sectional views of examples of wearable devices to detect inflammation coupled with a body portion in different states, a nominal state in FIG. 1A and an inflammation state in FIG. 1B, according to an embodiment of the present application;

FIG. 2 depicts an exemplary computer system, according to an embodiment of the present application;

FIG. 3 depicts a block diagram of one example of a wearable device to detect inflammation, according to an embodiment of the present application;

FIG. 4A depicts cross-sectional views of examples of a portion of the same body in three different dimensional states: a nominal dimension; a contracted dimension; and an inflammation dimension, according to an embodiment of the present application;

FIG. 4B depicts cross-sectional views examples of sensors in a wearable device to detect inflammation in contact with the body portions of FIG. 4A and generating signals, according to an embodiment of the present application;

FIG. 5 depicts a profile view of one example configuration for a wearable device to detect inflammation, according to an embodiment of the present application;

FIGS. 6A-6G depict examples of different configurations for a wearable device to detect inflammation, according to an embodiment of the present application;

FIGS. 7A-7B depict cross-sectional views of examples of different configurations for a wearable device to detect inflammation and associated sensor systems, according to an embodiment of the present application;

FIG. 7C depicts cross-sectional views of examples of a wearable device to detect inflammation and a sensor system in three different dimensional states related to a body portion being sensed, according to an embodiment of the present application;

FIG. 8A depicts a profile view of forces and motions acting on a user having a wearable device to detect inflammation, according to an embodiment of the present application;

FIG. 8B-8G depicts examples of activities of a user having a wearable device to detect inflammation, according to an embodiment of the present application;

FIG. 9 depicts a block diagram of sensor systems, data communication systems, data processing systems, wireless client devices, and data systems that may be coupled with and/or in communication with a wearable device to detect inflammation, according to an embodiment of the present application;

FIG. 10 depicts one example of a flow diagram for measuring, identifying, and remediating inflammation in a wearable device to detect inflammation, according to an embodiment of the present application;

FIG. 11 depicts a block diagram of an example of a system including one or more wearable devices to detect inflammation, according to an embodiment of the present application;

FIG. 12A depicts a profile view of one example of a wearable device to detect inflammation, according to an embodiment of the present application;

FIG. 12B depicts a cross-sectional view of one example of components in a wearable device to detect inflammation, according to an embodiment of the present application;

FIG. 12C depicts another profile view of another example of a wearable device to detect inflammation, according to an embodiment of the present application;

FIG. 13 depicts a block diagram of an example of a cycle of monitoring a user having a wearable device to detect inflammation and data inputs that may be used in a calculus for determining whether or not inflammation, contraction, or nominal states are indicated in the user, according to an embodiment of the present application;

FIG. 14 depicts one example of a flow diagram for passively determining a true resting heart rate (TRHR) of a user, according to an embodiment of the present application;

FIGS. 15A-15B depict two different examples of sensed data that may be relevant to passively determining a true resting heart rate (TRHR) of a user, according to an embodiment of the present application;

FIG. 16 depicts a block diagram of non-limiting examples of relevant sensor signals that may be parsed, read, scanned, and/or analyzed for passively determining a true resting heart rate (TRHR) of a user, according to an embodiment of the present application;

FIG. 17A depicts a block diagram of one example of sensor platform in a wearable device to passively detect fatigue of a user that includes a suite of sensors, according to an embodiment of the present application;

FIG. 17B depicts one example of a wearable device to passively detect fatigue of a user, according to an embodiment of the present application;

FIG. 17C depicts one example of speed of movement and heart rate as indicators of fatigue captured by sensors in communication with a wearable device to passively detect fatigue of a user, according to an embodiment of the present application;

FIG. 18 depicts examples of sensor inputs and/or data that may be sourced internally or externally in a wearable device to passively detect fatigue of a user, according to an embodiment of the present application; and

FIG. 19 depicts one example of a flow diagram for passively detecting fatigue in a user, according to an embodiment of the present application.

### DETAILED DESCRIPTION

Various embodiments or examples may be implemented in numerous ways, including as a system, a process, an apparatus, a user interface, or a series of program instructions on a non-transitory computer readable medium such as a computer readable storage medium or a computer network where the program instructions are sent over optical, electronic, or wireless communication links. In general, operations of disclosed processes may be performed in an arbitrary order, unless otherwise provided in the claims.

A detailed description of one or more examples is provided below along with accompanying drawing FIGS. The detailed description is provided in connection with such examples, but is not limited to any particular example. The scope is limited only by the claims and numerous alternatives, modifications, and equivalents are encompassed. Numerous specific details are set forth in the following description in order to provide a thorough understanding. These details are provided for the purpose of example and the described techniques may be practiced according to the claims without some or all of these specific details. For clarity, technical material that is known in the technical fields related to the examples has not been described in detail to avoid unnecessarily obscuring the description.

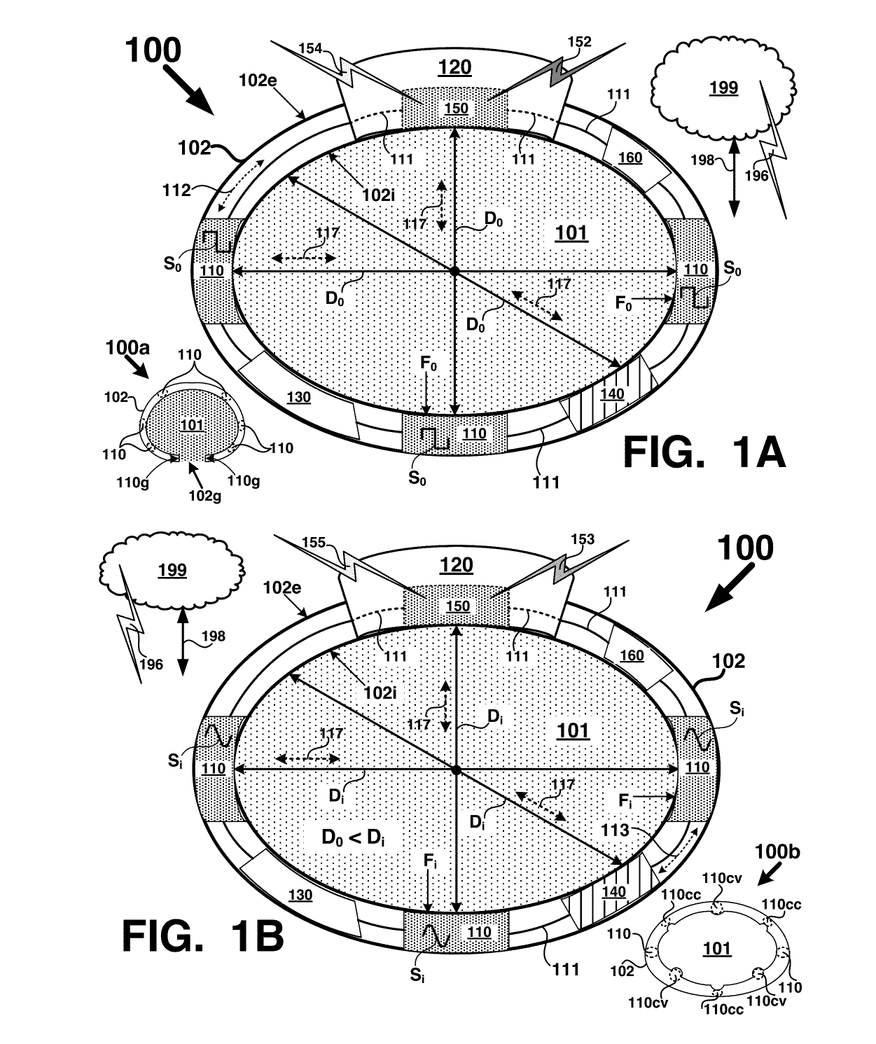

Reference is now made to FIGS. 1A-1B where cross-sectional views of examples of wearable devices to detect inflammation 100 (device 100 hereinafter) are coupled with a body portion in different states as will be described below. In FIGS. 1A-1B device 100 may include one or more sensors 110 for detecting/sensing force, pressure, or other metric associated with tissues of a body indicative of inflammation and/or contraction, for example. In that pressure may be defined a force per unit of area, hereinafter, the term force F will be used to describe the unit sensed by sensors 110 although one skilled in the art will understand that pressure or other metric may be interchangeably used in place of force F. Sensors 110 generate one or more signals S indicative of force acting on them via a coupling or contact with a body portion 101 of a user, such as a portion of an empennage, neck, torso, wrist, ankle, waist, or other area or portion of a body. In some examples, the body portion being sensed by sensors 110 is of a human body. In other examples, the body portion being sensed by sensors 110 is of a non-human body. For purposes of further explanation, a human body (e.g., of a user 800) will be used as a non-limiting example. Body portion 101 may comprise body tissue or tissues on a portion of a user body, such as the arms, legs, torso, neck, abdomen, etc. Sensors may be used to sense activity (e.g., biometric activity and related electrical signals) within the body tissue (e.g., body portion 101) or on a surface of the body tissue (e.g., a skin surface of body portion 101).

Device 100 may include other sensors for sensing environmental data, biometric data, motion data that may include little or no motion as in awake and resting or sleeping, just to name a few. Device 100 and some or all of its components may be positioned in a chassis 102 configured to be worn, donned, or otherwise connected with a portion of a user's body and configured to either directly contact some or all of the portion or to be positioned in close proximity to the portion. Device 100 may include a RF system 150 for wireless communication (152, 154, 153, 155) with external wireless systems using one or more radios which may be RF receivers, RF transmitters, or RF transceivers and those radios may use one or more wireless protocols (e.g., Bluetooth, Bluetooth Low Energy, NFC, WiFi, Cellular, broadband, one or more varieties of IEEE 802.11, etc.). Device 100 may include a user interface 120 such as a display (e.g., LED, OLED, LCD, touch screen or the like) or audio/video indicator system (e.g., speaker, microphone, vibration engine, etc.). As systemic inflammation may be a good to excellent indicator of a user's mood, device 100 may serve as a “mood ring” for a user's body. The display or one or more LED's (e.g., color LED's or RGB LED's) may be used to indicate mood as function of indication of inflammation, contraction, or nominal and those indications may be coupled with other biometric sensor readings (e.g., heart rate, heart rate variability, respiration, GSR, EMG, blood pressure, etc.) to indicate mood using one or more combinations of color, sound, or graphics/images presented on the display. In some examples, the user's mood may be displayed or otherwise presented for dissemination by the user, on an external device, such as a wireless client device (e.g., 680, 690, 999), the device 100 or both.

Device 100 may include a bus 111 or other electrically conductive structure for electrically communicating signals from sensors 110, other sensors, processor(s), data storage, I/O systems, power systems, communications interface, etc. Bus 111 may electrically couple other systems in device 100 such as power source 130 (e.g., a rechargeable battery), biometric sensors 140 (heart rate, body temperature, bioimpedance, respiration, blood oxygen, etc.), sensors of electrodermal activity on or below the skin (e.g., skin conductance, galvanic skin response—GSR, sensors that sense electrical activity of the sympathetic nervous system on the skin and/or below the skin, skin conductance response, electrodermal response, etc.), sensors that sense arousal, sensors for detecting activity of the sympathetic nervous system, electromyography (EMG) sensors, motion sensors 160 (e.g., single or multi-axis accelerometer, gyroscope, piezoelectric device), a compute engine (not shown, e.g., single-core or multiple-core processor, controller, DSP, ASIC, SoC, baseband processor, pP, pC, etc.), and data storage (not shown, e.g., Flash Memory, ROM, SRAM, DRAM, etc.).

Chassis 102 may have any configuration necessary for coupling with and sensing the body portion 101 of interest and chassis 102 may include an esthetic element (e.g., like jewelry) to appeal to fashion concerns, fads, vanity, or the like. Chassis 102 may be configured as a ring, ear ring, necklace, jewelry, arm band, head band, bracelet, cuff, leg band, watch, belt, sash, or other structure that may be worn or otherwise coupled with the body portion 101. Chassis 102 may include functional elements such as location of buttons, switches, actuators, indicators, displays, A/V devices, waterproofing, water resistance, vibration/impact resistance, just to name a few.

In FIGS. 1A-1B, device 100 is depicted in cross-sectional view and having an interior portion 102i in contact with the body portion 101 to be sensed by device 100 (e.g., sensed for inflammation, contraction, nominal state, or other). In FIG. 1A, the body portion 101 is depicted in a nominal state in which the body is not experiencing systemic inflammation or contraction (e.g., due to dehydration or other causation). In the nominal state, body portion 101 has nominal dimensions in various direction denoted as D₀ and a force F₀ indicative of the nominal state acts on sensors 101 which generate signal(s) indicative of the nominal state denoted as S₀. As will be described in greater detail below, state such as the nominal state, the contraction state, and the inflammation state may not be instantaneously determined in some examples, and those states may be determined and re-determined over time (e.g., minutes, hours, days, weeks, months) and in conjunction with other data inputs from different sources that may also be collected and or disseminated over time (e.g., minutes, hours, days, weeks, months).

In FIG. 1A, signals S₀ indicative of the nominal state (e.g., fluids in tissues of the user are not generating forces on sensors 101 indicative of inflammation and/or contraction) are electrically coupled over bus 111 to other systems of device 100 for analysis, processing, calculation, communication, etc. For example, data from signals S₀ may be wirelessly communicated (154, 152) to an external resource 199 (e.g., the Cloud, the Internet, a web page, web site, compute engine, data storage, etc.) and that data may be processed and/or stored with other data external to device 100, internal to device 100 (e.g., other sensors such as biometric sensors, motion sensors, location data) or both. Resource 199 may be in data communication (198, 196) with other systems and devices 100, using wired and/or wireless communications links. The determination that the state of the user is one that is the nominal state may not be an immediate determination and may require analysis and re-computation over time to arrive at a determination that one or more of D₀, F₀ or S₀ are indicative of the nominal state and the user is not experiencing systemic inflammation or contraction. Here, dimension D₀ may have variations in its actual dimension over time as denoted by dashed arrows 117. For example, due to changes in user data, environment, diet, stress, etc., a value for D₀ today may not be the same as the value for D₀ two months from today. As variation 117 may apply to the dimensions associated with contraction and inflammation as will be described below, that is, the dimensions may not be static and change over time as the user undergoes changes that are internal and/or external to the body.

In FIG. 1B, body portion 101 is depicted in an inflammation state where a dimension Dᵢ is indicative of systemic inflammation (e.g., increased pressure of fluids in tissues/cells of the user's body) and an inflammation force Fᵢ acts on sensors 110 to generate signal(s) Sᵢ and those signals may be electrically coupled over bus 111 to other systems of device 100 for analysis, processing, calculation, communication, etc. For example, data from signals Sᵢ may be wirelessly communicated (154, 152) to an external resource 199 as was described above in reference to FIG. 1A.

In FIGS. 1A-1B, chassis 102 of device 100 is depicted as having substantially smooth inner surfaces that contact the body portion 101 and completely encircling the body portion 101. However, actual shapes and configurations for chassis 102 may be application dependent (e.g., may depend on the body part the chassis 102 is to be mounted on) and are not limited to the examples depicted herein. Device 100a depicts an alternate example, where chassis 102 includes an opening or gap denoted as 102g and sensors 110 are positioned at a plurality of locations along the chassis 102 and other sensors denoted as 110g are positioned in the gap 102g. Here, as body part undergoes inflammation and its tissues expand, some of the expanded tissue may move into the gap 102g and exert force Fᵢ on sensors 110g and that force may be different (e.g., in magnitude) than the force Fᵢ exerted on sensors 110 along chassis 102. Accordingly, signals Sᵢ from sensors 110g and 110 may be different (e.g., in magnitude, waveform, voltage, current, etc.) and that difference may be used in the calculus for determining the inflammation state. Conversely, when body part is in the nominal state and/or contraction state, then portions of body part may not extend into the gap 102g and/or exert less Fᵢ on sensors 110g than on sensors 110 and that difference (e.g., in the signals Sᵢ from sensors 110g and 110) may be used in the calculus for determining which state the user is in (e.g., nominal, contraction, or inflammation).

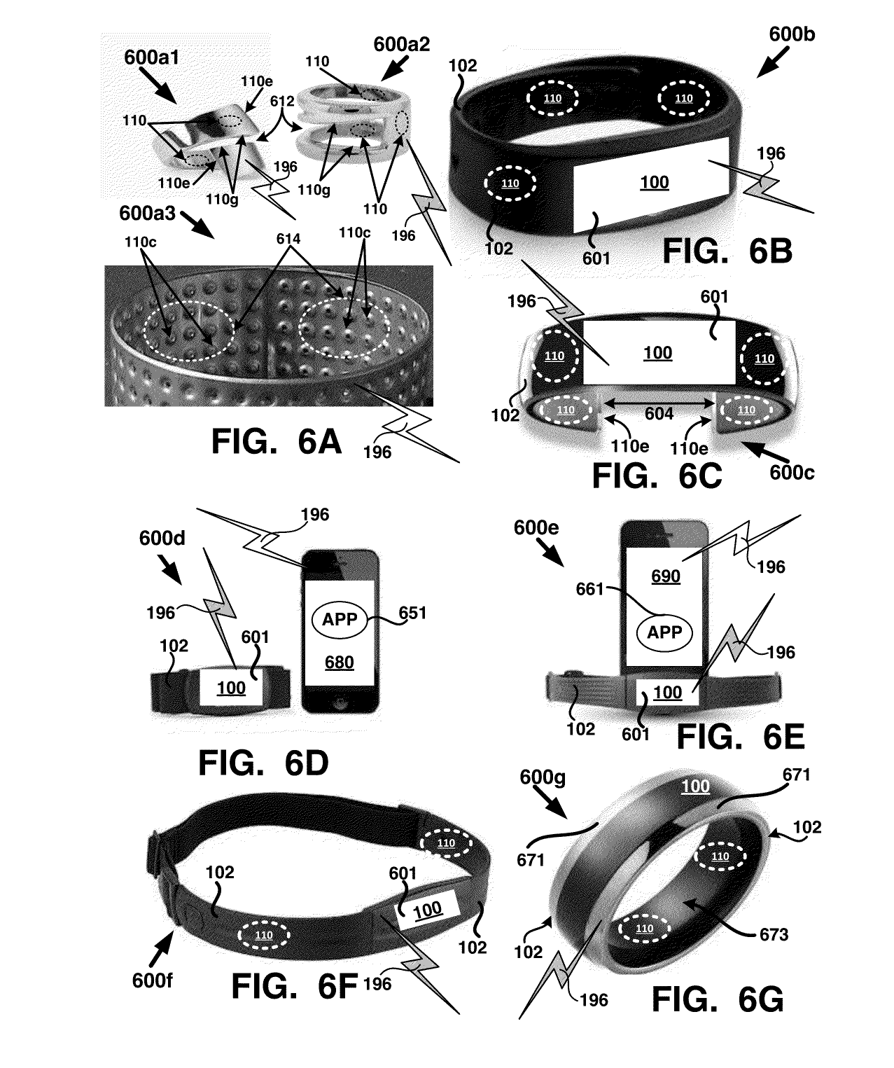

Device 100b depicts another alternate example, where chassis 102 includes along its interior portions that contact the body portion, one or more recessed or concave sensors 110cc and one or more protruding or convex sensors 100cv, and optionally one or more sensors 110. Here, when body portion 101 is undergoing inflammation, sensors 100cv may experience a higher Fᵢ due to its protruding/convex shape creating a high pressure point with the tissues urged into contact with it due to the inflammation. Sensors 100cc may experience a lower Fᵢ due to its recessed/concave shape creating a low pressure point with the tissues urged into contact with it due to the inflammation and/or those tissues not expanding into any or some of a volume created by the recessed/concave shape. Sensors 110 may experience a force Fᵢ that is in between that of sensors 110cv and 110cc. Accordingly, differences in signals Sᵢ from one or more of the sensors 110, 110cv, and 110cc may be processed and used in the calculus for determining which state the user is in as described above. Similarly, if body portion 101 is in the contraction state, sensors 110cc may experience little or no force Fᵢ because tissue may not contact their sensing surfaces, sensors 110cv may experience a force Fᵢ that is greater than the force Fᵢ experience by sensors 110 and the signals Sᵢ representative of those differences in force Fᵢ may be processed as described above to determine the users state. On the other hand, if body portion 101 is in the nominal state, sensors 110cc may experience little or no force Fᵢ because tissue may not contact their sensing surfaces, sensors 110cv may experience a force Fᵢ that is greater than the force Fᵢ experience by sensors 110 and the signals Sᵢ representative of those differences in force Fᵢ may be processed as described above to determine the users state. The processing along with other data inputs may be used to determine if the signals Sᵢ are more indicative of the contraction state or the nominal state, as those states may have similar characteristics for signals Sᵢ. Alternate chassis and sensor 110 locations will be described in greater detail below in regards to FIGS. 6A-6G. Shapes for sensors 110cv and/or 110cc may be formed by slots, grooves, ridges, undulations, crenulations, dimples, bumps, domes (inward and/outward facing), gaps, spacing's, channels, canals, or other structures and are not limited to the structures depicted herein.

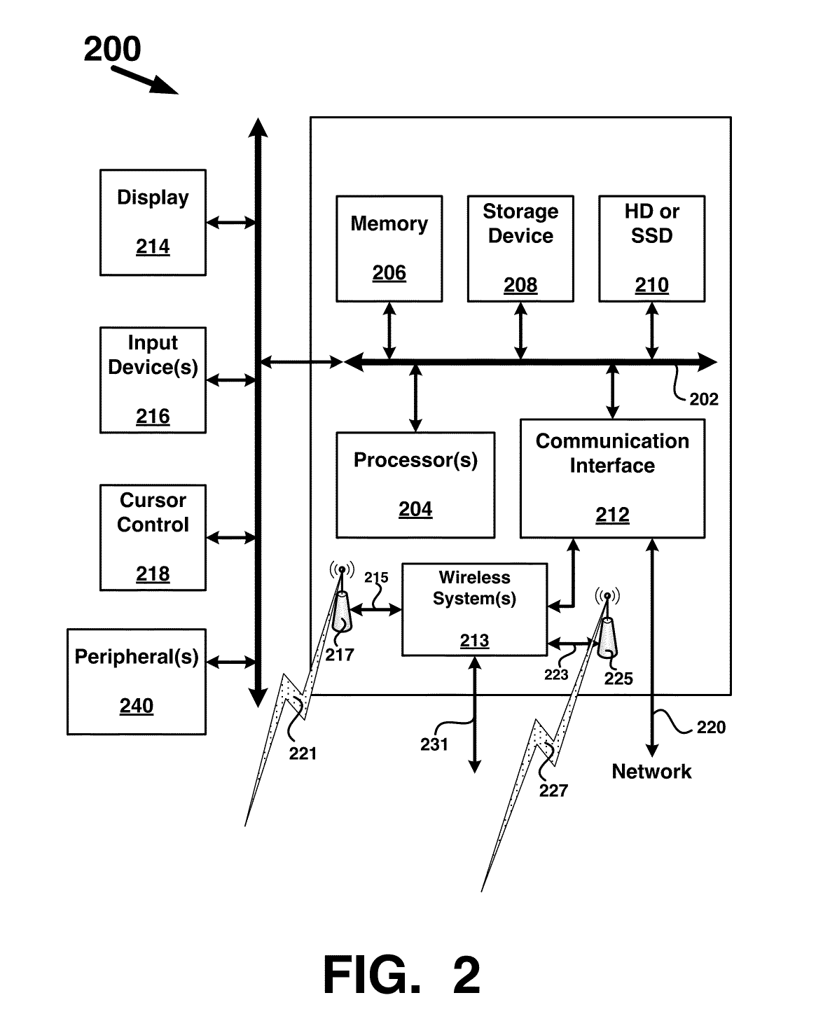

FIG. 2 depicts an exemplary computer system 200 suitable for use in the systems, methods, and apparatus described herein. In some examples, computer system 200 may be used to implement circuitry, computer programs, applications (e.g., APP's), configurations (e.g., CFG's), methods, processes, or other hardware and/or software to perform the above-described techniques. Computer system 200 includes a bus 202 or other communication mechanism for communicating information, which interconnects subsystems and devices, such as one or more processors 204, system memory 206 (e.g., RAM, SRAM, DRAM, Flash), storage device 208 (e.g., Flash Memory, ROM), disk drive 210 (e.g., magnetic, optical, solid state), communication interface 212 (e.g., modem, Ethernet, one or more varieties of IEEE 802.11, WiFi, WiMAX, WiFi Direct, Bluetooth, Bluetooth Low Energy, NFC, Ad Hoc WiFi, HackRF, USB-powered software-defined radio (SDR), WAN or other), display 214 (e.g., CRT, LCD, OLED, touch screen), one or more input devices 216 (e.g., keyboard, stylus, touch screen display), cursor control 218 (e.g., mouse, trackball, stylus), one or more peripherals 240. Some of the elements depicted in computer system 200 may be optional, such as elements 214-218 and 240, for example and computer system 200 need not include all of the elements depicted.

According to some examples, computer system 200 performs specific operations by processor 204 executing one or more sequences of one or more instructions stored in system memory 206. Such instructions may be read into system memory 206 from another non-transitory computer readable medium, such as storage device 208 or disk drive 210 (e.g., a HD or SSD). In some examples, circuitry may be used in place of or in combination with software instructions for implementation. The term “non-transitory computer readable medium” refers to any tangible medium that participates in providing instructions to processor 204 for execution. Such a medium may take many forms, including but not limited to, non-volatile media and volatile media. Non-volatile media includes, for example, Flash Memory, optical, magnetic, or solid state disks, such as disk drive 210. Volatile media includes dynamic memory (e.g., DRAM), such as system memory 206. Common forms of non-transitory computer readable media includes, for example, floppy disk, flexible disk, hard disk, Flash Memory, SSD, magnetic tape, any other magnetic medium, CD-ROM, DVD-ROM, Blu-Ray ROM, USB thumb drive, SD Card, any other optical medium, punch cards, paper tape, any other physical medium with patterns of holes, RAM, PROM, EPROM, FLASH-EPROM, any other memory chip or cartridge, or any other medium from which a computer may read.

Instructions may further be transmitted or received using a transmission medium. The term “transmission medium” may include any tangible or intangible medium that is capable of storing, encoding or carrying instructions for execution by the machine, and includes digital or analog communications signals or other intangible medium to facilitate communication of such instructions. Transmission media includes coaxial cables, copper wire, and fiber optics, including wires that comprise bus 202 for transmitting a computer data signal. In some examples, execution of the sequences of instructions may be performed by a single computer system 200. According to some examples, two or more computer systems 200 coupled by communication link 220 (e.g., LAN, Ethernet, PSTN, wireless network, WiFi, WiMAX, Bluetooth (BT), NFC, Ad Hoc WiFi, HackRF, USB-powered software-defined radio (SDR), or other) may perform the sequence of instructions in coordination with one another. Computer system 200 may transmit and receive messages, data, and instructions, including programs, (e.g., application code), through communication link 220 and communication interface 212. Received program code may be executed by processor 204 as it is received, and/or stored in a drive unit 210 (e.g., a SSD or HD) or other non-volatile storage for later execution. Computer system 200 may optionally include one or more wireless systems 213 in communication with the communication interface 212 and coupled (215, 223) with one or more antennas (217, 225) for receiving and/or transmitting RF signals (221, 227), such as from a WiFi network, BT radio, or other wireless network and/or wireless devices, for example. Examples of wireless devices include but are not limited to: a data capable strap band, wristband, wristwatch, digital watch, or wireless activity monitoring and reporting device; a smartphone; cellular phone; tablet; tablet computer; pad device (e.g., an iPad); touch screen device; touch screen computer; laptop computer; personal computer; server; personal digital assistant (PDA); portable gaming device; a mobile electronic device; and a wireless media device, just to name a few. Computer system 200 in part or whole may be used to implement one or more systems, devices, or methods that communicate with transponder 100 via RF signals (e.g., RF System 135) or a hard wired connection (e.g., data port 138). For example, a radio (e.g., a RF receiver) in wireless system(s) 213 may receive transmitted RF signals (e.g., 154, 152, 153, 155 or other RF signals) from wearable device 100 that include one or more datum (e.g., sensor system information) related to nominal state, inflammation, contraction, temperature, temporal data, biometric data, forces, motion, or other events in a user's body. Computer system 200 in part or whole may be used to implement a remote server or other compute engine in communication with systems, devices, or method for use with the transponder 100 as described herein. Computer system 200 in part or whole may be included in a portable device such as a smartphone, tablet, or pad. The portable device may be carried by an emergency responder or medical professional who may use the datum transmitted Tx 132 by transponder 100 and received and presented by the computer system 200 to aid in treating or otherwise assisting the user wearing the transponder 100.

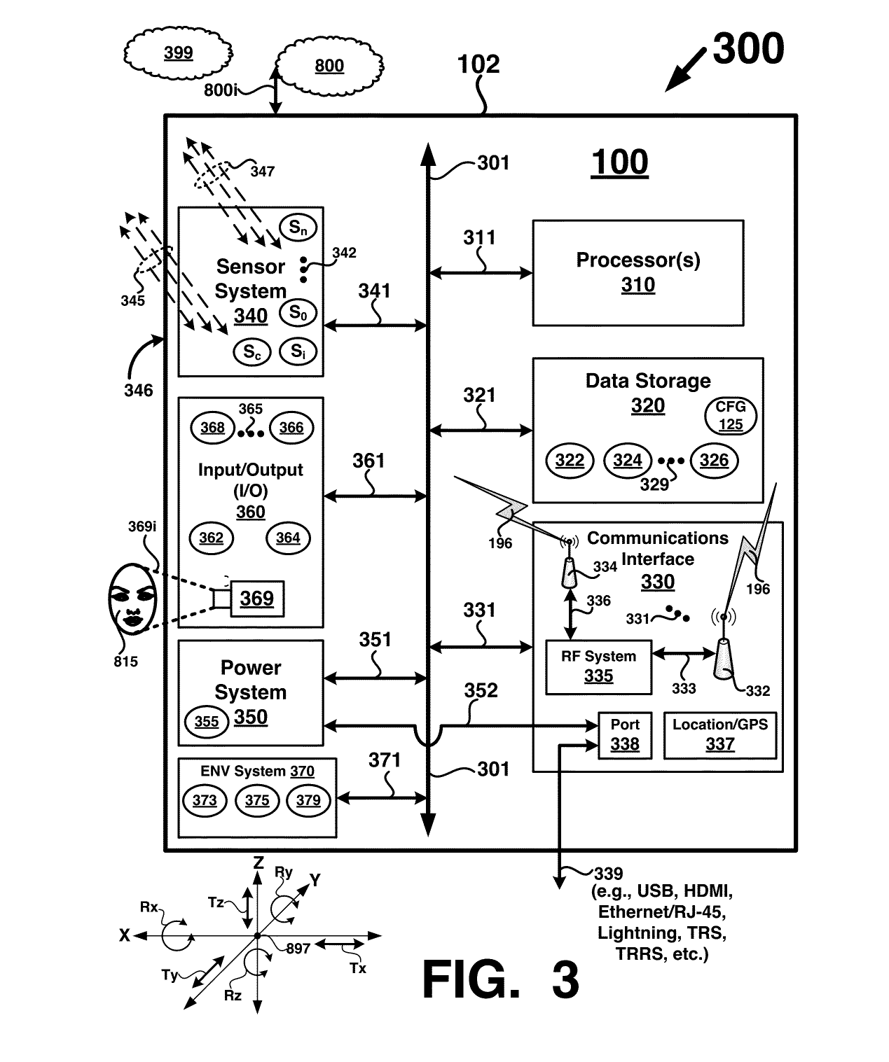

Turning now to FIG. 3 where a block diagram of one example 300 of a wearable device to detect inflammation 100 is depicted. In example 300, device 100 may include but is not limited to having one or more processors, a data storage unit 320, a communications interface 330, a sensor system 340, a power system 350, an input/output (I/O) system 360, and an environmental sensor 370. The foregoing are non-limiting examples of what may be included in device 100 and device 100 may include more, fewer, other, or different systems than depicted. The systems of device 100 may be in communication (311, 321, 331, 341, 351, 352, 361, 371) with a bus 301 or some other electrically conductive structure. In some examples, one or more systems of device 100 may include wireless communication of data and/or signals to one or more other systems of device 100 or another device 100 that is wirelessly linked with device 100 (e.g., via communications interface 330).

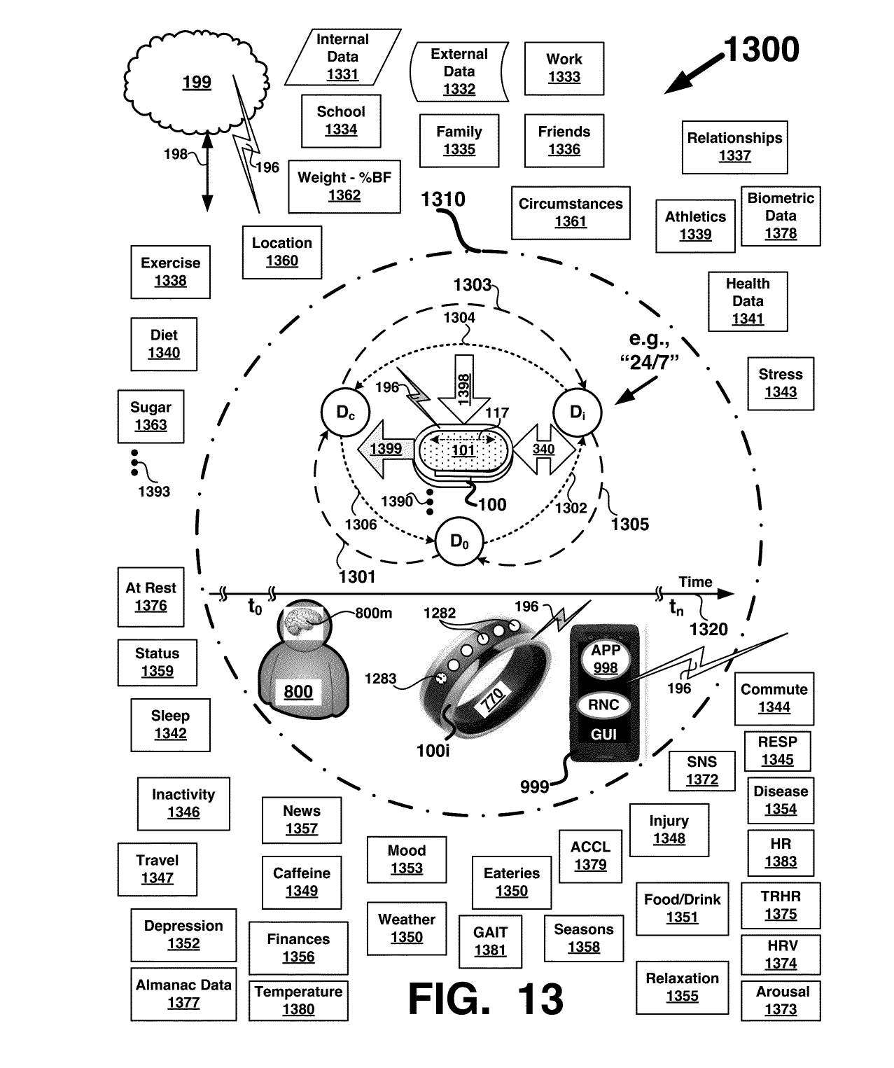

The various systems may be electrically coupled with a bus 301 (e.g., see bus 111 in FIGS. 1A-1B). Sensor system 340 may include one or more sensors that may be configured to sense 345 an environment 399 external 346 to chassis 102 such as temperature, sound, light, atmosphere, etc. In some examples, one or more sensors for sensing environment 399 may be included in the environmental system 370, such as a sensor 373 for sound (e.g., a microphone or other acoustic transducer), a light sensor 375 (e.g., an ambient light sensor, an optoelectronic device, a photo diode, PIN diode, photo cell, photo-sensitive device 1283 of FIG. 13, etc.), and an atmospheric sensor 378 (e.g., a solid state, a semiconductor, or a metal oxide sensor). Sensor system 340 may include one or more sensors for sensing 347 a user 800 that is connected with or otherwise coupled 800i with device 100 (e.g., via a portion of chassis 102) and those sensors may include the aforementioned biometric and other sensors. Sensor system 340 includes one or more of the sensors 110, 110cv, 110cc for generating the signals S₀, Sᵢ, S_{c} as described above. Signals from other sensors in sensor system 340 are generically denoted as Sₙ and there may be more signals Sₙ than depicted as denoted by 342. Processor(s) 301 may include one or more of the compute engines as described above (e.g., single-core or multiple-core processor, controller, DSP, ASIC, SoC, baseband processor, μP, μP, etc.). Computation, analysis or other compute functions associated with signals from sensor system 340 may occur in processor 310, external to device 100 (e.g., in resource 199) or both. Data and results from external computation/processing may be communicated to/from device 100 using communications interface 330 via wireless 196 or wired 339 communications links. Sensor system 340 may include one or more motion sensors (e.g., single-axis or multi-axis accelerometers, gyroscopes, vibration detectors, piezoelectric devices, etc.) that generate one or more of the signals Sₙ, and those signals Sₙ may be generated by motion and/or lack of motion (e.g., running, exercise, sleep, rest, eating, etc.) of the user 800, such as translation (Tx, Ty, Tz) and/or rotation (Rx, Ry, Rz) about an X-Y-Z axes 897 of the users body during day-to-day activities. In some examples, the motion signals Sₙ may be from sensors external to device 100 (e.g., from other devices 100, fitness monitors, data capable strap bands, exercise equipment, smart watches or other wireless systems), internal to device 100 or both.

Data storage unit 320 may include one or more operating systems (OS), boot code, BIOS, algorithms, data, user data, tables, data structures, applications (APP) or configurations (CFG) denoted as 322-326 that may be embodied in a non-transitory computer readable medium (NTCRM) that may be configured to execute on processor 310, an external processor/compute engine (e.g., resource 199) or both. There may be more or fewer elements in data storage unit 320 (DS 320 hereinafter) as denoted by 329. As one example, DS 320 may comprise non-volatile memory, such as Flash memory. CFG 125 may be a configuration file used for configuring device 100 to communicate with wireless client devices, other devices 100, with wireless access points (AP's), resource 199, and other external systems. Moreover, CFG 125 may execute on processor 310 and include executable code and/or data for one or more functions of device 100. CFG 125 may include data for establishing wireless communications links with external wireless devices using one or more protocols including but not limited to Bluetooth, IEEE 802.11, NFC, Ad Hoc WiFi, just to name a few, for example.

Communications interface 330 may include a RF system 335 coupled with one or more radios 332, 336 for wireless 196 communications, an external communications port 338 for wired communications with external systems. Port 338 may comprise a standard interface (e.g., USB, HDMI, Lightning, Ethernet, RJ-45, TRS, TRRS, etc.) or proprietary interface. Communications interface 330 may include a location/GPS unit for determining location of the device 100 (e.g., as worn by the user 800) and/or for gathering location/GPS data from an external source or both. The one or more radios 332, 336 may communicate using different wireless protocols. There may be more or fewer radios and or systems in RF system 335 as denoted by 331.

Power system 350 may supply electrical power at whatever voltages and current demands required by systems of device 100 using circuitry and/or algorithms for power conditioning, power management, power regulation, power savings, power standby, etc. One or more power sources 355 may be coupled with power system 350, such as rechargeable batteries (e.g., Lithium Ion or the like), for example.

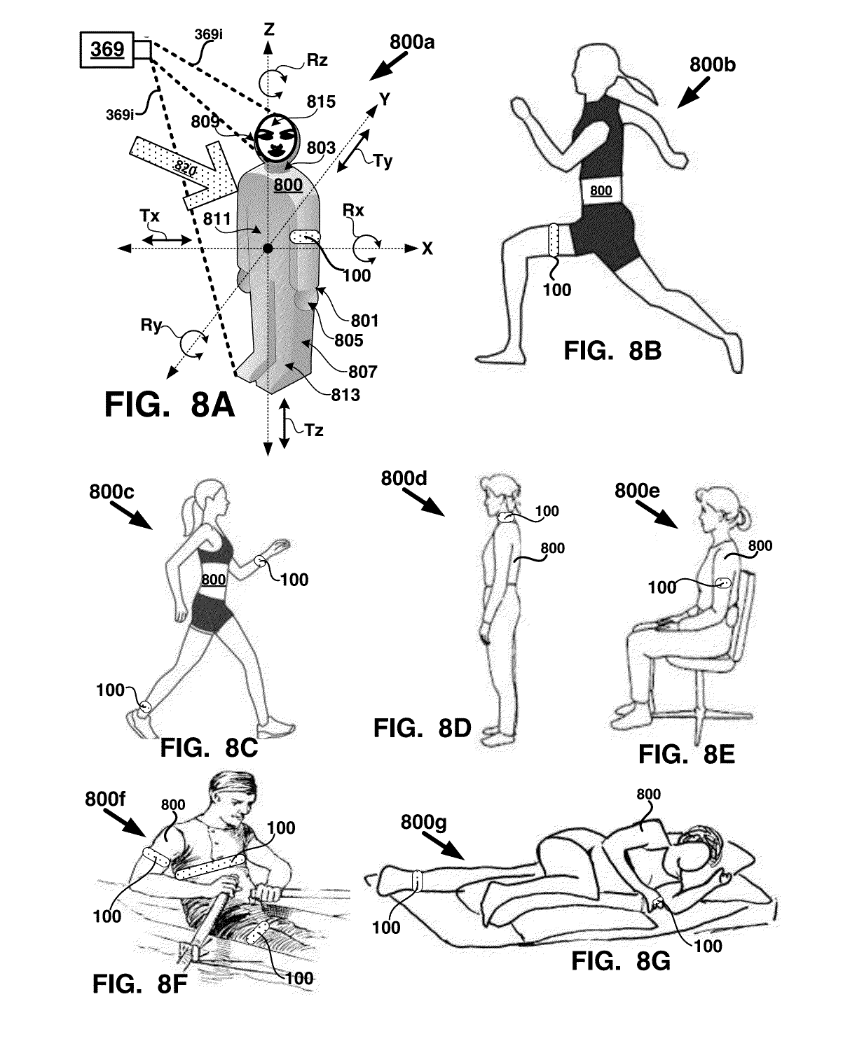

I/O system 360 may include one or more hardware and/or software elements denoted as 362-368 of which there may be more or fewer than depicted as denoted by 365. Those elements may include but are not limited to a display or other user interface (e.g., 120 of FIGS. 1A-1B), a microphone, a speaker, a vibration engine (e.g., a buzzer or the like), indicator lights (e.g., LED's), just to name a few. I/O system 360 may communicate data to/from the communications interface 330 to other systems in device 100 (e.g., via bus 301 or 111), for example. An image capture device 369 may be included in I/O system 360, sensor system 340 or both. Image capture device 369 (e.g., video or still images) may be used to image 369i facial expressions and/or micro-expressions on a face 815 of a user 800. Image capture device 369 (e.g., video or still images) may be used to image 369i a posture of the user 800's body (e.g., see 369 and 369i in FIG. 8A). Hardware and/or software may be used to process captured image 369i data and generate an output signal that may be used in determining fatigue, stress, systemic inflammation, contraction, or other conditions of user 800's emotional, mental or physical state. Signals from image capture device 369 may be treated as one form of sensor signal, regardless of the system in device 100 that the image capture device is positioned in or associated with.

As a person skilled in the art will recognize from the previous detailed description and from the drawing FIGS. and claims set forth below, modifications and changes may be made to the embodiments of the present application without departing from the scope of this present application as defined in the following claims.

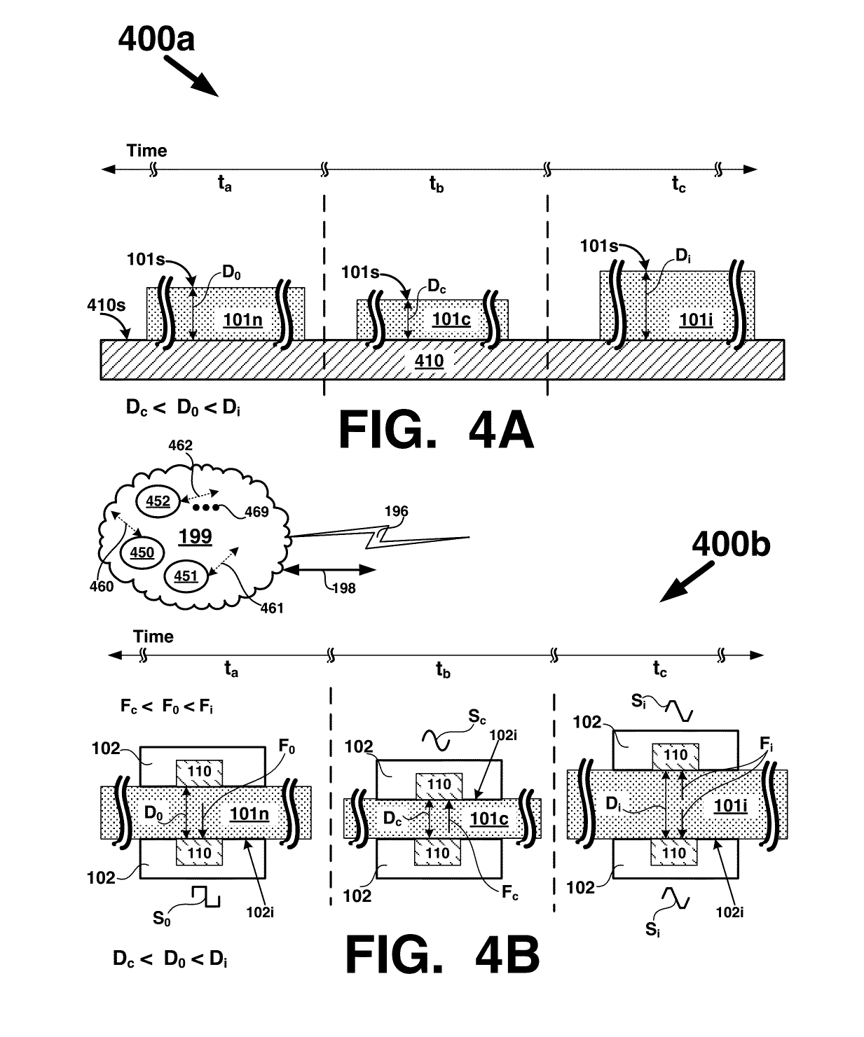

Moving on to FIG. 4A where cross-sectional views of examples 400a of a portion of the same body in three different dimensional states comprised of a nominal dimension 101n, a contracted dimension 101c, and an inflammation dimension 101i are depicted. For purposes of explanation, assume that the body portion depicted is resting on a flat and rigid surface 410, such as a table top or the like such that a distance from a top 410s of the surface 410 to a top 101s of the body portions in the different states (101n, 101c, 101i) may be accurately measured. In order of Time from the left of the drawing sheet to the right of the drawing sheet, at a time interval tₐ, a dimension D₀ (e.g., height or thickness from 410s to 101s) of the nominal body 101n is measured, and subsequent measurements taken at later time intervals t_{b} and t_{c} yield dimensions of D_{c} and Dᵢ respectively for contracted body portion 101c and inflamed body portion 101i respectively. Here, D_{c}<D₀<Dᵢ. Over the different time intervals (tₐ, t_{b}, t_{c}) the dimensions of the body portion changed as conditions internal to and/or external to the users body changed. These changes in dimension may continuously vary over Time with the dimensions sometimes being nominal, sometimes being contracted, and sometimes being inflamed.

Referring now to FIG. 4B were cross-sectional views examples 400b of sensors 110 in a wearable device to detect inflammation 100 in contact 102i with the body portions of FIG. 4A and generating signals indicative of the aforementioned dimensions of the body portions in different states (101n, 101c, 101i). During time interval tₐ, a dimension D₀ exerts a force F₀ on sensor 110 which generates a signal S₀ indicative of the nominal state for body portion 101n during time interval tₐ. Similarly, later time intervals t_{b} and t_{c} yield dimensions of D_{c} and Dᵢ, exerted forces F_{c} and Fᵢ, and generated signals S_{c} and Sᵢ respectively for contracted body portion 101c and inflamed body portion 101i during those intervals of Time. Here, D_{c}<D₀<Dᵢ and F_{c}<F₀<Fᵢ. The differences in waveform shapes for the generated signals Sₙ, S_{c} and Sᵢ are only for purposes of illustration and actual waveforms may be different than depicted. Generated signals Sₙ, S_{c} and Sᵢ may or may not follow the relationship S_{c}<S₀<Sᵢ and actual signal magnitudes may be application dependent and may not be linear or proportional to force exerted on sensors 110 by the body portions depicted. In FIG. 4B, the dimension may continuously vary over Time with the dimensions sometimes being nominal, sometimes being contracted, and sometimes being inflamed as was described above. As the nominal, contracted, and inflamed dimensions change with Time, device 100 and/or other devices in communication with device 100 may repeatedly update and retrieve signal data or other data associated with the states from a source such as DS 320 and/or an external resource (e.g., 199). For this example 400b, the signal and/or other data for the three states may be repeatedly updated, stored, retrieved or otherwise accessed from resource 199 as denoted by dashed arrows 460-462 for nominal state related data 450, contracted state related data 451, and inflamed state related data 452. The aforementioned changes in dimension over Time are repeatedly sensed and compared with other data to calculate the actual state of the user (i.e., nominal, contracted, inflammation). Therefore an instantaneous or sudden change in any one of the signals (Sₙ, S_{c} and Sᵢ) from sensors 110 does not automatically indicate an accurate determination of state in the absence of other information and data used in the calculus for determining state. Resource 199 may include additional data as denoted by 469 and that data, as will be described above, may be used along with the signal data to calculate state.

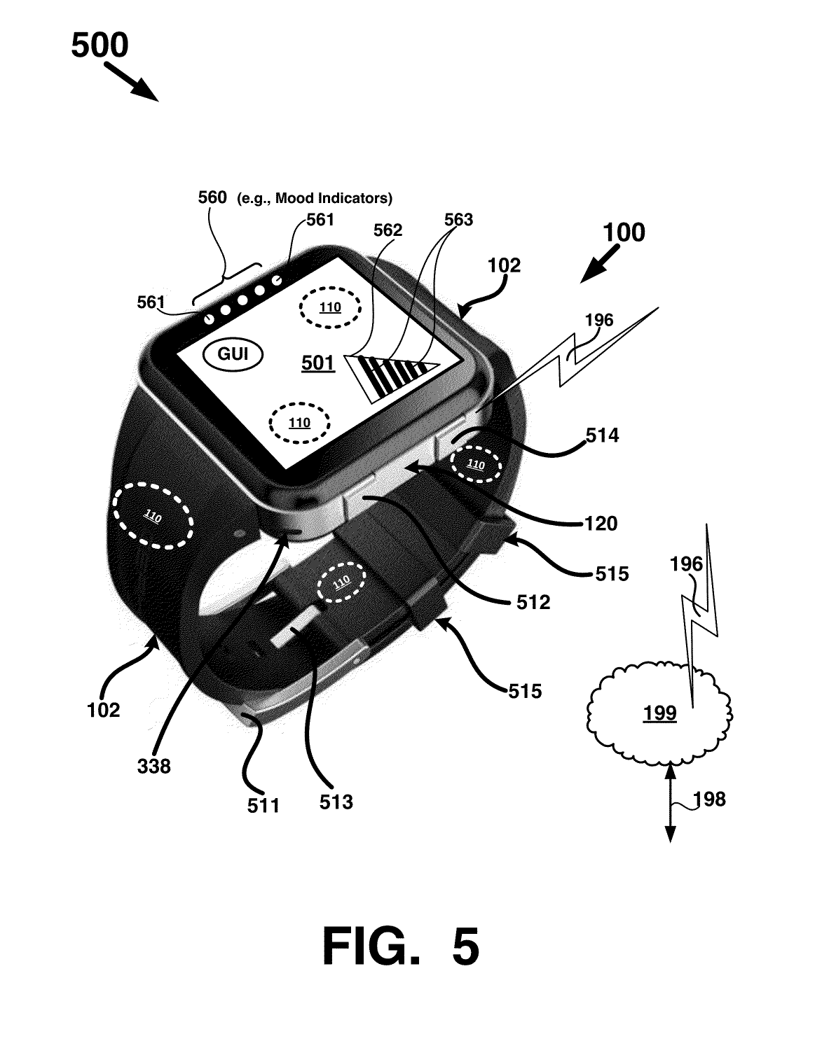

Moving on to FIG. 5 where a profile view of one example 500 of a configuration for a wearable device to detect inflammation 100 may include a watch band, strap or the like as part of chassis 102, a buckle 511, tongue 513, loops 515, and a user interface 120 that may include buttons 512 and 514, a display 501, a port 338 for wired communication links (e.g., 198) and/or charging an internal power source such as a rechargeable battery, and a RF system for wireless 196 communications (e.g., with resource 199 or other devices 100). Device 100 may include a plurality of the sensors 110 disposed at various positions about the strap 102 and user interface 120 as denoted in dashed outline. Upon donning the device 100, a user may set baseline tension or tightness of the device 100 (e.g., about a wrist) such that one or more portions of the users body are coupled or otherwise connected with the sensors 110. In that motion of the user, the device 100, the tension of strap 102 and other factors may change a magnitude of the force (F_{c}, F₀, Fᵢ) exerted by body tissues against the sensors 110, the above mentioned repeated measurements may be used to arrive at correct states over time when used with other data as described above. As will be described in greater detail below, device 100 may include one or more indicators 561 that may be used to visually display a mood of the user (e.g., of the user's body), as denoted by mood indicators 560. One or more indicator devices such as a LED may be used for indicator 561, for example. Alternatively or in addition to mood indicators 560, display 501 may include a GUI or other form of information presentation that includes a graphic or icon displaying the user mood, such as mood indicator 562 which is depicted as a plurality of black bars 563, where more bars 563 may indicate a better mood than fewer bars 563. Similarly, a better mood may be indicated by more of the indicators 561 lighting up than fewer indicators 561 lighting up.

FIGS. 6A-6G depict additional examples of different configurations for a wearable device to detect inflammation 100. In FIGS. 6A and 6G, device 100 may be configured as a ring to be worn on one of the digits of a hand (e.g., fingers or thumb) of a user or optionally on one of the toes of a foot of the user. Swelling of the tissues of the hand, the fingers of the toes are typical when systemic inflammation occurs (e.g., a feeling of puffiness in the hands/fingers) and those body portions may be ideal locations to position device 100 for measuring inflammation. In examples 600a1 and 601a2 of FIG. 6A, device 100 is configured to have one or more grooves or spirals denoted as 612. Sensors 110 are disposed at a plurality of locations as depicted in dashed line; however, sensors 110g are disposed at 612 so that tissue from the fingers that expand outward during inflammation may enter into the groove/spiral 612 and exert force (e.g., Fᵢ) on sensors 110g. Sensors 110g may measure force as described above or some other metric such as capacitance or GSR, for example. In example 600a3, device 100 includes a plurality of dimples similar to the sensors 110cv and 110cc of FIG. 1B positioned at an interior portion (e.g., 102i) of chassis 102 as denoted by dashed regions 614. The dimples may be concave, convex or both. Depending on the state of the body, dimples that are concave may experience a different force than dimples that are convex and signals from those concave and convex dimples may be used to determine the aforementioned states.

In FIG. 6G, device 100 has a chassis configured as a ring. Here, chassis 102 includes a rigid structure 671 and a deformable structure 673, and sensors 110 are disposed at various locations within the deformable structure 673. As the body portion the ring is positioned on, expands and contracts due to tissue fluids etc. (e.g., D_{c}, D₀, Dᵢ), the deformable structure 673 is compressed upon expansion of the tissue and relaxed upon contraction of the tissues. Forces imparted to the deformable structure 673 by the expansion or contraction may be mechanically coupled with the sensors 110 to generate the signals (S_{c}, S₀, Sᵢ) from the exerted forces (F_{c}, F₀, Fᵢ).

In FIG. 6B, device 100 may be configured to have a chassis 102 formed as a band that may be worn on a wrist or ankle of a user, for example. Band 102 may lack a buckle or other fastening structure such as that depicted in FIG. 5 and may instead be made of semi-flexible materials that retain their shape after being wrapped around the body portion to be sensed (e.g., the wrist or ankle). Sensors 110 may be positioned at locations along band 102 where tissue contact (e.g., 101) may be most effectively coupled with the sensors 110. Devices 100 in FIGS. 6B-6F, may optionally include a display 601.

In FIG. 6C, device 100 includes a chassis 102 that may be configured as a bracelet or armband, for example. Band 102 includes an opening 604 which may be used to ease insertion and removal of the body portion (e.g., an arm or ankle) to be sensed by sensors 110 that are disposed at various locations on an interior portion of band 102. Sensors 110e may be positioned at opposing edges of opening 604 and may be configured to sense forces from tissue that expands into the opening 604 due to inflammation as was describe above in reference to FIG. 6A.

In FIGS. 6D-6F, device 100 may be configured as a band (600d) or waist band or chest band (600e, 600f). In FIGS. 6D and 6E, device 100 may be wirelessly linked 196 (e.g., via WiFi or Bluetooth) with a client device (680, 690) that includes an application (APP 651, APP 661) which may be presented on display 601 in the form of a graphical users interface (GUI) through which a user may configure, control, query, command, and perform other operations on device 100. Client device (680, 690) may replace or work in conjunction with resource 199 and/or device 100 to analyze, process, and calculate the states as described above.

The depictions in FIGS. 6A-6G are non-limiting example of devices 100 and other configurations are possible. The devices 100 depicted in FIGS. 6A-6G may all have wireless communication links, wired links or both. In other examples, a user may wear one or more of the devices 100 depicted in FIGS. 6A-6G or elsewhere as described herein, and those devices 100 may be in wireless communication with one another and with resource 199 or other external sources or systems. Data (e.g., from sensor system 340) may be collected and wirelessly transmitted by a plurality of the devices 100 and one or more of those devices 100 may process, analyze, calculate or perform other operations on that data either individually, with an external system (e.g., 199 or other) or both.

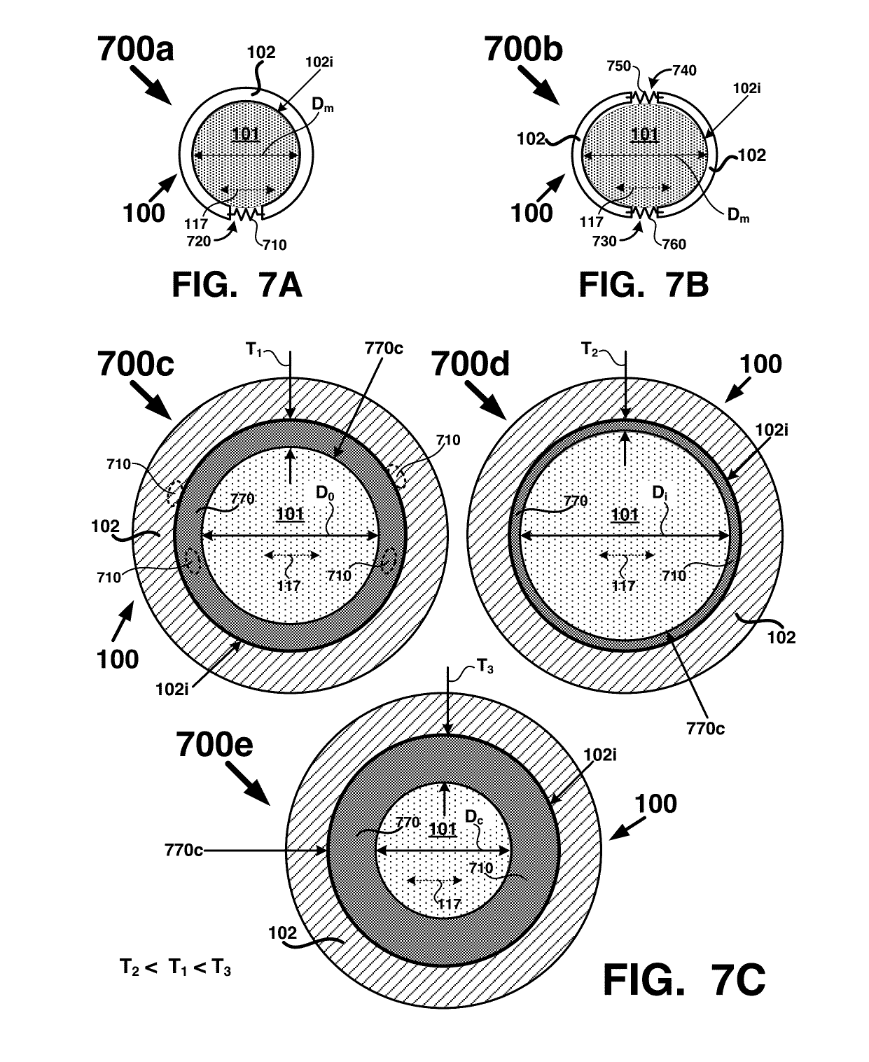

Turning now to FIGS. 7A-7B where cross-sectional views of examples 700a and 700b of different configurations for a wearable device to detect inflammation 100 and associated sensor systems 710 are depicted. In FIG. 7A, chassis 102 includes an opening 720 and a sensor 710 positioned in the opening and coupled with chassis 102. A body portion 101 having a dimension D_{M} (e.g., some diameter of a finger, wrist, ankle, etc.) may be inserted into an interior portion of chassis 102 and in contact with interior surfaces of the chassis 102. Expansion and/or contraction of the body portion 101 generate the aforementioned forces that may cause the chassis 102 to expand primarily at the opening 720 in response to forces caused by expansion of the body portion 101, or cause the chassis 102 to contract primarily at the opening 720 in response to forces caused by contraction of the body portion 101 as denoted by dashed arrow 117. Sensor 710 may generate a signal indicative of expansion, contraction, or nominal status based on forces acting on the sensor 710 or on some other metric sensed by sensor 710. Sensor 710 may include but is not limited to a strain gauge, a piezoelectric device, a capacitive device, a resistive device, or an inductive device. As one example, as a piezoelectric device, sensor 710 may generate a signal of a first magnitude and/or waveform when forces generated by expansion of body portion 101 causes the opening to expand outward and imparting stress or strain (e.g., tension or stretching) to the piezoelectric device causing the signal Sᵢ to be generated. On the other hand, sensor 710 may generate a signal of a second magnitude and/or waveform when forces generated by contraction of body portion 101 causes the opening to contract inward and imparting stress or strain (e.g., compression, squeezing, or deformation) to the piezoelectric device causing the signal S_{c} to be generated. Sensor 710 may generate the nominal signal Sₙ when forces acting on it over time generate signals that are within a range of values not indicative of inflammation (e.g., expansion of opening 720) or of dehydration or other (e.g., contraction of opening 720). In other examples, sensor 710 may be a variable capacitance-based, variable resistance-based, or variable inductance-based device that changes its capacitance, resistance or inductance in response to being stretched or compressed.

In FIG. 7B, chassis 102 includes a plurality of openings (730, 740) and sensors (750, 760) positioned in those openings and coupled with chassis 102. The position of the plurality of openings (730, 740) in chassis 102 may be different than depicted and there may be more than two openings. Sensor 750 and 760 need not be identical types or configurations of sensors and have different operating characteristics and may output different signals in response to expansion, contraction, or nominal forces. As described above in respect to FIG. 7A, expansion and contraction of openings (730, 740) cause signals Sᵢ or S_{c} to be generated. As describe above nominal signal S₀ may be determined over time for each sensor 750 and 760. Here, sensor 750 may experience different applied forces than sensor 760 in response to expansion and contraction of body portion 101 or in response to a nominal condition of body portion 101. Over time, signals Sᵢ and/or S_{c} from sensor 750 and 760 may be sampled or otherwise processed to determine if body portion 101 is inflamed or contracted. For example, if over a period of time (e.g., approximately 9 hours) signals from both sensors (750, 760) are indicative of a trend of increasing generated signal strength, that trend may be analyzed to determine inflammation is present in body portion 101 and likely elsewhere in the body of the user 800. Previous nominal signal S₀ values may be used to validate the upward trending signals (e.g., Sᵢ) from both sensors (750, 760) that are indicative of inflammation. Similarly, for downward trending signals from both sensors (750, 760), a determination may be made (e.g., including using previous nominal signal S₀ values) that body portion 101 has shrunken due to dehydration or other cause. A voting protocol may be invoked when there is an unresolvable difference between the signals from both sensors (750, 760) such that sensor 750 indicates contraction and sensor 760 indicates expansion. If chassis 102 is configured to include three or more sensors disposed in three or more gaps, then the voting protocol may determine inflammation or contraction when a majority of the sensors indicate inflammation or contraction (e.g., 2 out of 3 sensors or 4 out of 5 sensors), for example.

Referring now to FIG. 7C where three examples 770c-700e depict another example configuration for device 100. In example 700c, body portion 101 is inserted or otherwise coupled with a flexible structure 770 in which one or more sensors 710 may be coupled with or may be disposed in flexible structure 770. Chassis 102 may surround or otherwise be in contact with or coupled with the flexible structure 770 along an interior 102i of the chassis 102. Body portion 101 may be completely surrounded by or otherwise in contact with or coupled with the flexible structure 770 along an interior 770c of the flexible structure 770. Flexible structure 770 may be made from a variety of materials that are flexible and/or may be repeatedly expanded and contracted in shape when pressure or force is exerted on the material. Examples include but are not limited to Sorbothane, Foam, Silicone, Rubber, a balloon or bladder filled with a fluid such as a gas or liquid or viscous material such as oil, and a diaphragm, just to name a few.

In example 700c body portion 101 is depicted inserted into device 100 and having dimension D₀ for the nominal state so that an approximate thickness between 102i and 770c is denoted as T₁. As body portion 101 expands and contracts, flexible structure 770 may also expand and contract as denoted by dashed arrow 117. One or more of the sensors 710 may be positioned within the flexible structure 770 so that as the flexible structure 770 expands or contracts, forces from the expansion or contraction may couple with the sensor 710 and the sensor 710 may generate a signal (e.g., S₀, S_{c}, Sᵢ) responsive or otherwise indicative of the force being applied to it. Other locations for sensor 710 may be within an aperture or other opening formed in chassis 102 and operative to allow forces from the expansion or contraction of 770 to couple with the chassis mounted sensor 710. Both non-limiting examples for the configurations for sensor 710 are depicted in example 700c and there may be more or fewer sensors 710 than depicted and other sensor locations may be used.

In example 700d the body portion 101 has expanded from dimension D₀ to Dᵢ dimension such that approximate thickness between 102i and 770c has reduced from T₁ to T₂. Here, sensor(s) 710 may generate signal Sᵢ indicative of possible inflammation. In contrast, in example 700e the body portion 101 has contracted from dimension D₀ (or other dimension such as Dᵢ) to D_{c} dimension such that approximate thickness between 102i and 770c has increased from T₁ (or other thickness such as T₂) to T₃. Here, sensor(s) 710 may generate signal S_{c} indicative of possible contraction (e.g., dehydration). The examples 700c-700e and configurations for device 100 depicted in FIG. 7C may be used to implement a device 100 such as the rings depicted in FIGS. 6A and 6G or bracelets in FIGS. 6B-6D, for example. As one example, flexible structure 770 may be used for the deformable structure 673 of example 600g in FIG. 6G.

Attention is now directed to FIG. 8A where a profile view of forces 820 and motions (Tx, Ty, Tz, Rx, Ry, Rz) acting on a user 800 having (e.g., wearing) a wearable device to detect inflammation 100 are depicted. In example 800a, the user 800 may be in motion and/or the aforementioned forces may be acting on user 800's body such that motion signals may be generated by sensors in sensory system 340 in device 100 or other devices user 800 may have that may be configured to generate motion signals that may be communicated to device 100 and/or another system (e.g., 199) for purposes of analysis, calculation, data collection, coaching, avoidance, etc. Although device 100 is depicted being worn about an arm (e.g., around the biceps) of user 800, actual locations on the body of user 800 are not limited to the location depicted. Other non-limiting locations may include but are not limited to wrist 801 (e.g., a bracelet or band for device 100), neck 803, hand 805 (e.g., a ring for device 100), leg 807, head 809, torso 811, and ankle 813, for example.

Movement of user 800's body or parts of the body (e.g., limbs, head, etc.) relative to the X-Y-Z axes depicted may generate motion signals and/or force signals (e.g., S₀, S_{c}, Sᵢ) due to translation (T) and rotation (R) motions (Tx, Ty, Tz, Rx, Ry, Rz) about the X-Y-Z axes. As will be described in relation to subsequent FIGS., force signals (e.g., S₀, S_{c}, Sᵢ) caused by motion of user 800 or imparted to user 800 by another actor (e.g., a bumpy ride in a car), may need to be cancelled out, excluded, disregarded, or otherwise processed so as to eliminate errors and/or false data for force signals (e.g., S₀, S_{c}, Sᵢ), that is, determining the state (e.g., nominal, contracted, inflamed) may require signal processing or other algorithms and/or hardware to separate actual data for force signals (e.g., S₀, S_{c}, Sᵢ) from potentially false or erroneous data caused by motion or other inputs that may cause sensors (110, 710, 750, 760, 710) to output signals that are not related to or caused by changes in state of the body portion being sensed by device 100 and its various systems.

Accordingly, motion signals from sensor system 340 or other systems in device 100 or other devices may be used to filter out non-state related force signals (e.g., S₀, S_{c}, Sᵢ) in real-time as the signals are generated, post signal acquisition where the signals are stored and later operated on and/or analyzed or both, for example. To that end, example 800b of FIG. 8B depicts user 800 running with device 100 worn about a thigh of the user 800. User 800 may be prone to overexerting herself to the point where inflammation may result from over training, for example. While user 800 is running, forces such as those depicted in FIG. 8A may act on sensors (e.g., 110) in device 100 and some of that force may generate signals from the sensors that may require filtering using motion signals from motion sensors or the like to cull bad or false signal data from actual state related signal data. As one example, a cadence of user 800 as she runs may generate motion signals that have a pattern (e.g., in magnitude and/or time) that may approximately match the cadence of user 800. Sensors (e.g., 110) coupled with the body portion (e.g., thigh were device 100 is positioned) to be sensed may also experience forces generated by the cadence (e.g., footfalls, pumping of the arms, etc. associated with running) and signals generated by the sensors may also approximately match the cadence of user 800. The amount of signal data generated by the sensors during the running may be highly suspect as legitimate state related signals because of the repetitive nature of those signals due to the cadence and the simultaneous occurrence of motion signals having a similar pattern or signature as the cadence. Generated force signals (e.g., S₀, S_{c}, Sᵢ) may be ignored when the user 800 is running, may be compared with the motion signals or otherwise filtered using data from the motion signals to derive more accurate state related signals, which may indicate that inflammation is occurring (e.g., body portion 101 may show a trend of expansion) during the running due to an excessive workout, an injury, etc. On the other hand, during the running the filtered/processed state signals may indicate contraction is occurring because user 800 has not been properly hydrating her-self (e.g., drinking water) during the running and some trend of shrinkage of the body portion 101 is indicated. In other examples, taking all signal inputs that may be necessary to filter out bad data, etc., the state related signals may indicate no significant deviation of the body portion 101 from the nominal state (e.g., the body portion has not indicated a trend towards expansion or contraction).

FIGS. 8C-8G depict examples 800c-800g of other activities of a user 800 that may or may not require additional signal processing and/or analysis to determine accurate state related signals. As one example, going from left to right in FIGS. 8C-8E, the amount of additional signal processing that may be necessary for example 800c where the user 800 is walking may be more than is required for example 800d where the user 800 is standing, and may be even less for example 800e where the user 800 is sitting down. In contrast, example 800f depicts a user 800 rowing and that activity may require additional signal processing as compared with example 800g where the user 800 is resting or sleeping. Example 800g also depicts one example of a multi-device 100 scenario where user 800 has two of the devices 100, one device 100 on a finger of the right hand and another device 100 on the left ankle. In the multi-device 100 scenario there may be a plurality of the devices 100 (e.g., see 800c, 800f, 800g). Those devices 100 may operate independently of one another, one or more of those devices 100 may work in cooperation or conjunction with one another, and one of those devices 100 may be designated (e.g., by user 800 or an APP 661, 651) as or act as a master device 100 that may control and/or orchestrate operation of the other devices 100 which may be designated (e.g., by user 800 or an APP) as or act as subordinate devices 100. Some or all of the devices in a multi-device 100 scenario may be wirelessly linked with one another and/or with an external system or devices (e.g., 199, 680, 690, 200). A single device 100 or multiple devices 100 may be used to gather data about a user's activity, such as motion profiles of how the user 800 walks or runs, etc. In example 800c, devices 100 may be used to gather historical data or other data on user 800's gait while in motion. Gait detection may include but is not limited to detecting accelerometry/motion associated with heel strikes, forefoot strikes, midfoot strikes, limb movement, limb movement patterns, velocity of the body, movement of different segments of the body, pumping and/or movement of the arms, just to name a few. Historical data and/or norms for the user, motion profiles, or other data about the user may be used as data inputs for processing/analysis of accelerometry, motion signals, or other sensor signals or data (e.g., location/GPS data). Gait detection and/or monitoring may be used with or without historical data to determine one or more of biometric data (e.g., true resting heart rate, heart rate variability), physiological and/or psychological state (e.g., fatigue), etc., and those determinations, including indications I/C/N, may made be made without active input or action taking by user 800, that is, the determinations may be made by device(s) 100 automatically without user intervention (e.g., a passive user mode). Moreover, those determinations and resulting outputs (e.g., reports, notifications, coaching, avoidance, user mood, etc.) may be handled by device(s) 100 on a continuous basis (e.g., 24 hours a day, seven days a week—24/7).

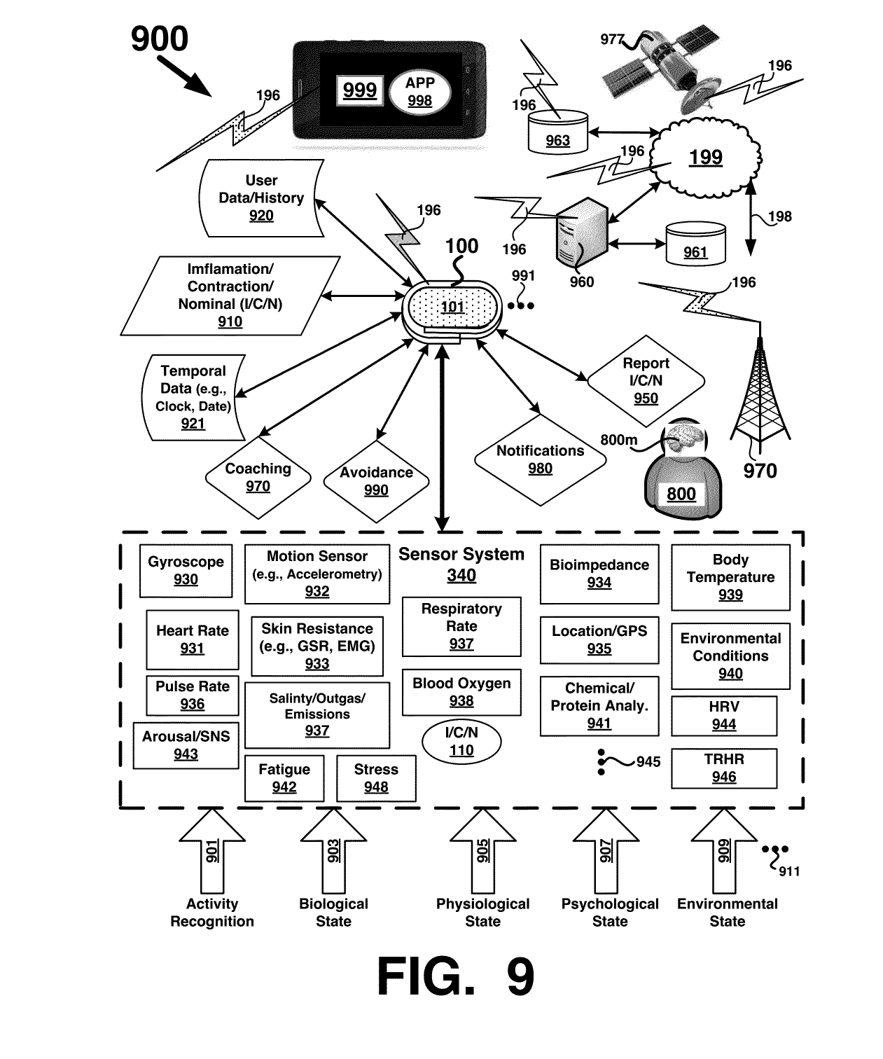

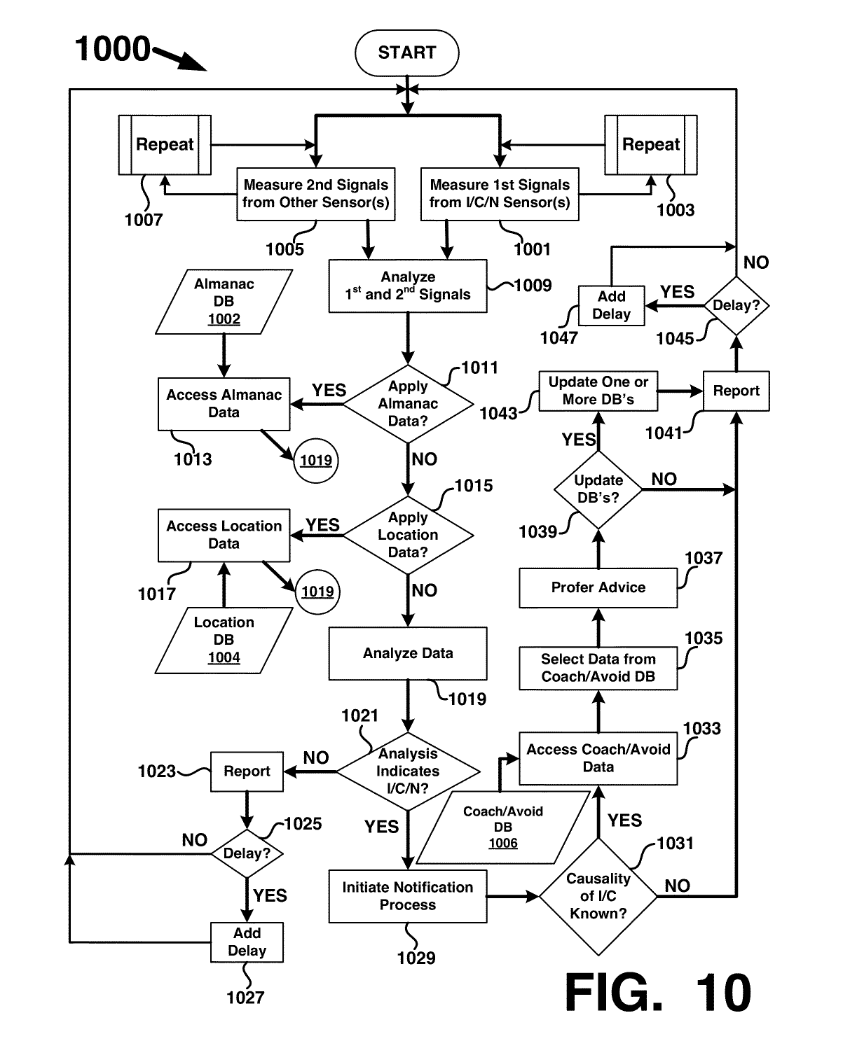

Referring now to FIG. 9 where a block diagram 900 of sensor systems, data communication systems, data processing systems, wireless client devices, and data systems that may be coupled with and/or in communication with a wearable device 100 to detect inflammation are depicted. Device 100 may use its various systems to collect and/or process data and/or signals from a variety of sensors and other systems. As one example, accurate determination of state (e.g., nominal, contracted, inflammation) of the user 800 may require a plurality of sensors and their related signals as depicted for sensor system 340 which may sense inputs including but not limited to activity recognition 901 (e.g., rest, sleep, work, exercise, eating, relaxing, chores, etc.), biological state 903 (e.g., biometric data), physiological state 905 (e.g., state of health of user 800's body), psychological state 907 (e.g., state of mental health of user 800's mind 800m), and environmental state 909 (e.g., conditions in environment around the user 800). There may be more of fewer inputs than depicted as denoted by 911 and some of the inputs may be interrelated to one another. There may be more devices 100 than depicted as denoted by 991 and those devices 100 may be wirelessly linked 196 with one another.

Sensor system 340 may include but is not limited to sensors such as the aforementioned sensor(s) I/C/N 110 (e.g., for sensing force applied by body portion 101), a gyroscope 930, motion sensor 932 (e.g., accelerometry using an accelerometer), bioimpedance 934, body temperature 939, hear rate 931, skin resistance 933 (e.g., GSR), respiratory rate 937, location/GPS 935, environmental conditions 940 (e.g., external temperature/weather, etc.), pulse rate 936, salinity/outgas/emissions 937 (e.g., from skin of user 800), blood oxygen level 938, chemical/protein analysis 941, fatigue 942, stress 948, true resting heart rate (TRHR) 946, heart rate variability (HRV) 944, just to name a few. As will be described below, sensor system 340 may include sensors for detecting electrical activity associated with arousal activity in the sympathetic nervous system denoted as Arousal/SNS 943. GSR 933 and bioimpedance 934 are non-limiting examples of SNS related sensors. Device 100 may use some or all of the sensor signals from sensor system 340. In some applications, one or more of the sensors in sensor system 340 may be an external sensor included in another device (e.g., another device 100) and signal data from those external sensors may be wirelessly communicated 196 to the device 100 by the another device or by some other system such as 199, 963, 970, 960, 977 (e.g., a communications and/or GPS satellite), for example. Other inputs and/or data for device 100 may include temporal data 921 (e.g., time, date, month, year, etc.), user data/history 920 which may comprise without limitation any information about and/or of and concerning the user 800 that may relate to health, diet, weight, profession/occupation (e.g., for determining potential stress levels), activities, sports, habits (e.g., the user 800 is a smoker), and status (e.g., single, married, number of children, etc.), and data 910 from (e.g., from sensor(s) 110) related to the states of inflammation, contraction, and nominal, just to name a few. Processing, analysis, calculation, and other compute operations may occur internal to systems of device 100, external to device 100 or both. The aforementioned compute operations may be offloaded to external devices/systems or shared between device 100 and other devices and systems. For example, client device 999 may include an APP 998 and processor(s) for performing the compute operations.

Device 100 based on analysis of at least a portion of the data may issue one or more notifications 980, may issue coaching (e.g., proffer advice) 970, may report 950 the state (I/C/N) to user 800, and may issue avoidance 990 (e.g., proffer advice as to how to avoid future reoccurrences of inflammation, fatigue, stress, etc.). A data base may be used as a source for coaching data, avoidance data or both. Report 950 may comprise an indication of whether or not the user 800 has systemic inflammation, is experiencing contraction (e.g., related to dehydration), or is in a nominal state. Notifications 980 may comprise a wide variety of data that may be communicated to user 800 including but not limited to notice of stress levels indicated by some of the data that was processed, steps user 800 may take to remediate inflammation, contraction or other conditions, locations for food, drink or other dietary needs of the user 800, just to name a few. As one example, if user 800 is experiencing inflammation caused by high dose of sugar (e.g., from eating ice cream), then using location data 935, device 100 may notify user 800 of a nearby coffee shop where a caffeinated drink may be obtained as an anti-inflammatory. Coaching 970 may include but is not limited to advising and/or offering suggestions to the user 800 for changing behavior or to improve some aspect of the wellbeing of the user 800. As one example, if user 800 is bicycling 25 miles each day non-stop (e.g., without sufficient breaks for water or rest), coaching 970 may advise user 800 that inflammation being detected by device 100 may be the result of overdoing his/her exercise routine and may suggest more stops along the route to rest and hydrate or to reduce the speed at which the user 800 is peddling the bicycle to reduce stress to the muscles, etc.

The Report 950, Notifications 980, Coaching 970, and Avoidance 990 may be presented to user 800 in any number of ways including but not limited to one or more of a display on device 100 or a client device (e.g., 999), an email message, a text message, an instant message, a Tweet, a posting to a blog or web page, a message sent to a professional or social media page, and an audio message, just to name a few. The information/data in Report 950, Notifications 980, and Coaching 970, and the method in which the information/data is communicated may be as varied and extensive as hardware and/or software systems may allow and may evolve or change without limitation. Although I/C/N is depicted in regards to 910 and 950, other conditions affection user 800 such as true resting heart rate (TRHR), fatigue (e.g., due to stress or other) may also be included in one or more of the user data history 920, the coaching 970, the avoidance 990, the notifications 980, the reports 950, as will be described below.

Now, FIG. 10 depicts one example of a flow diagram 1000 for measuring, identifying, and remediating inflammation in a wearable device to detect inflammation 100. At a stage 1001 and/or a stage 1005, sensor signals (e.g., from sensor system 340) may be measured, with a first set of signals measured from sensors for inflammation/contraction/nominal states (e.g., 110) and a second set of signals from other sensors (e.g., motion and biometric). Stages 1001 and 1005 may occur in series, in parallel, synchronously or asynchronously. For example, second set of signals from motion sensors may be measured at the same time as the first set of signals from sensor(s) 110. The stage 1001, the stage 1005 or both may repeat at stages 1003 and 1007 respectively. Repeating at the stages 1003 and 1007 may comprise continuing to measure the first and/or second signals or restarting the measurement of the first and/or second signals.

At a stage 1009 analysis may be performed on the first and second signals to determine which of the three states the user may be in and to correct data errors (e.g., to remove false I/C/N data caused by motion). Stages 1001 and/or 1005 may be repeating (1003, 1007) while stage 1009 is executing or other stages in flow 1000 are executing.

At a stage 1011 a decision may be made as to whether or not to apply almanac data to the analysis from the stage 1009. If a YES branch is taken, then flow 1000 may access the almanac data at a stage 1013 and stage 1013 may access an almanac data base 1002 to obtain the almanac data. Almanac DB 1002 may include historical data about a user of device 100, data about the environment in which the user resides and other data that may have bearing on causing or remediating inflammation and/or contraction and may be used to guide the user back to a nominal state. Flow 1000 may transition to another stage after execution of the stage 1013, such as a stage 1019, for example. If the NO branch is taken, then flow 1000 may continue at a stage 1015 where a decision to apply location data (e.g., from GPS tracking of a client device associated with the user—e.g., a smartphone or tablet). If a YES branch is taken, then flow 1000 may transition to a stage 1017 where location data is accessed. Accessed data may be obtained from a location database which may include a log of locations visited by the user and associations/connections of those locations with user behavior such as locations of eateries frequented by the user, locations associated with events that may cause stress in the user (e.g., commute or picking up the kids from school), and other forms of data without limitation. Flow 1000 may transition to another stage after execution of the stage 1017, such as a stage 1019, for example. If the NO branch is taken, then flow 1000 may transition to a stage 1019 where some or all of the data compiled from prior stages may be analyzed and flow may transition from the stage 1019 to a stage 1021.

At the stage 1021 a determination may be made as to whether or not the analysis at the stage 1019 indicates inflammation, contraction, or nominal state (I/C/N). In some applications the stage 1021 may only determine if inflammation (I) or contraction (C) are indicated and the nominal state (N) may not figure into the decision. If a NO branch is taken, then flow 1000 may proceed to a stage 1023 where a report of the indication at the stage 1021 may be generated. At a stage 1025 a decision as to whether or not to delay the report generated at the stage 1023 may be made with the YES branch adding delay at a stage 1027 or the NO branch transitioning flow 1000 to another stage, such as stages 1005 and/or 1001. The NO branch from the stage 1021 may mean that the data as analyzed thus far may be inconclusive of an indication of I/C/N and the return of flow 1000 back to stages 1005 and/or 1001 may comprise reiterating the prior stages until some indication of I/C/N occurs. The adding of delay at the stage 1027 may be to operative to add wait states or to allow signals received by sensor system 340 to stabilize, for example.

If the YES branch is taken from the stage 1021, then flow 1000 may transition to a stage 1029 where a notification process may be initiated and flow 1000 may transition to a stage 1031 where a determination as to whether or not a cause of inflammation or contraction is known. If a NO branch is taken, then flow 1000 may transition to a stage 1041 where delay at a stage 1045 may optionally be added as described above at a stage 1047 and flow 1000 may cycle back to stages 1005 and/or 1001. Analysis at the stage 1019, determining the indication at the stage 1021, the reporting at the stage 1023 may include delaying taking any actions or proceeding to other stages in flow 1000 until a certain amount of time has elapsed (e.g., wait states or delay) to allow device 100 to re-calculate, re-analyze or other steps to verify accuracy of data or signals used in those stages. If a plurality of devices 100 are worn by user 800, then a device 100 indicating inflammation or contraction may query other devices 100 to determine if one or more of those devices 100 concur with it by also indicating inflammation or contraction, for example.

If a YES branch is taken from the stage 1031, then flow may transition to a stage 1033 where coaching and/or avoidance data may be accessed (e.g., from coaching/avoidance DB 1006 or other). Accessing at the stage 1033 may include an address for data in a data base (e.g., 1006) that matches a known cause of the inflammation I or the contraction C. At a stage 1035 data from the data base (e.g., coaching and/or avoidance DB 1006) is selected and at a stage 1037 advice based on the selection at the stage 1035 is proffered to the user in some user understandable form such as audio, video or both.

At a stage 1039 a decision to update a database may be made, such as the data sources discussed in FIG. 9, may be updated. If a YES branch is taken, then flow 1000 may transition to a stage 1043 where one or more data bases are updated and flow may transition to the stage 1041 as described above. Here, flow 1000 may allow for data sources used by device 100 to be updated with current data or data used to analyze whether or not the user is undergoing I or C. Some or all of the stages in flow 1000 may be implemented in hardware, circuitry, software or any combination of the foregoing. Software implementations of one or more stages of flow 1000 may be embodied in a non-transitory computer readable medium configured to execute on a general purpose computer or compute engine, including but not limited to those described herein in regards to FIGS. 1A-1B, 2, 3, 6A-6G, 9 and 13. Stages in flow 1000 may be distributed among different devices and/or systems for execution and/or among a plurality of devices 100.

Hardware and/or software on device 100 may operate intermittently or continuously (e.g., 24/7) to sense the user 800's body and external environment. Detection and/or indication of (I/C/N) (e.g., using flow 1000 and/or other facilities of device 100) may be an ongoing monitoring process where indications, notifications, reports, and coaching may continue, be revised, be updated, etc., as the device 100 and its systems (e.g., sensor system 340) continue to monitor and detect changes in the user 800's body, such as in the dimension of the body portion 101. Systemic inflammation may trend upward (e.g., increasing Dᵢ over time), trend downward (e.g., decreasing Dᵢ over time), transition back to nominal (e.g., D₀), transition to contracted (e.g., D_{c}), or make any number of transitions within a state or between states, for example.

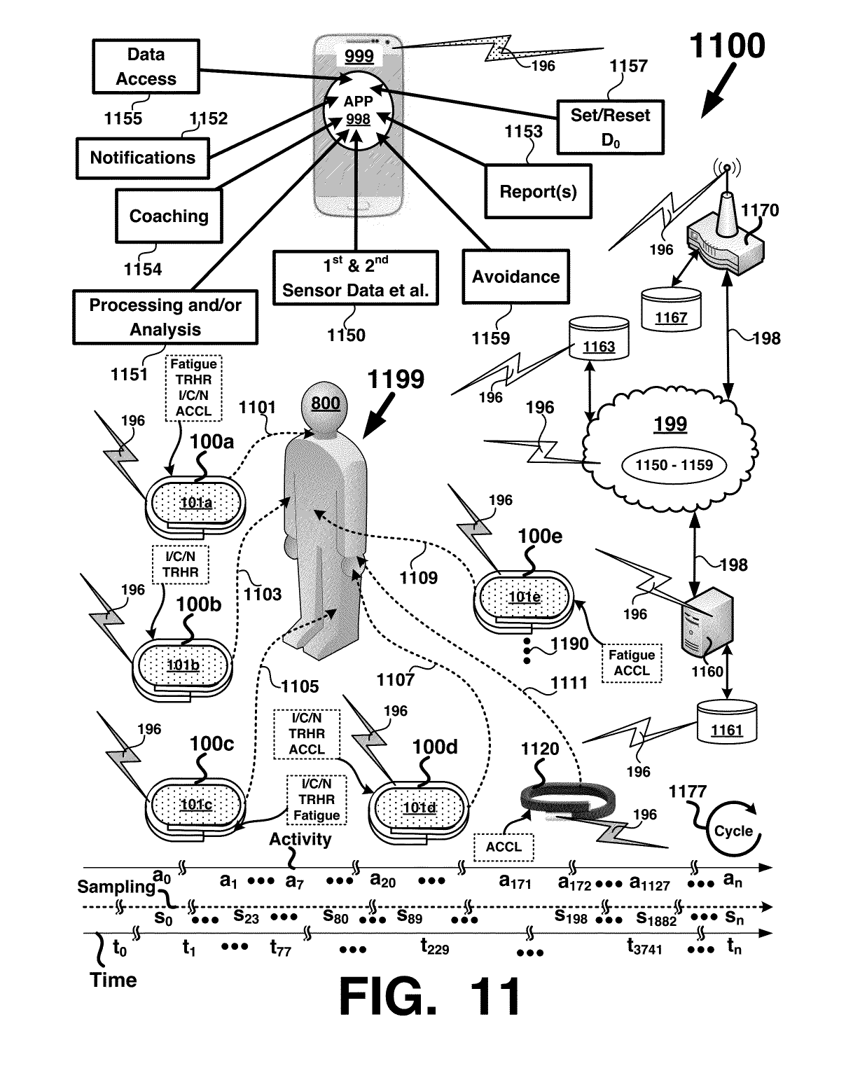

Moving along to FIG. 11 where a block diagram of an example of a system 1100 including one or more wearable devices 100a-100e to detect inflammation are depicted. Here system 1100 may include but is not limited to one or more client devices 999 (e.g., a wireless client device such as a smartphone, smart watch, tablet, pad, PC, laptop, etc.), resource 199, data storage 1163, server 1160 optionally coupled with data storage 1161, wireless access point (AP) 1170, network attached storage (NAS) 1167, and one or more devices 100 denoted as wearable devices 100a-100e. Some or all of the elements depicted in FIG. 11 may be in wireless communications 196 with one another and/or with specific devices. In some examples, some of the devices 100a-100e may be configured differently than other of the devices 100a-100e. There may be more or fewer devices 100a-100e as denoted by 1190.

User 800 may wear or otherwise don one or more of the devices 100a-100e for detecting inflammation at one or more different locations 1101-1109 on user 800's body, such as a neck body portion 101a for device 100a, an arm body portion 101b for device 100b, a leg body portion 101c for device 100c, a finger body portion 101d for device 100d, and a torso body portion 101e for device 100e, for example. User 800 may also don one or more other wearable devices such as a data capable strap band, a fitness monitor, a smart watch or other devices and sensor data from one or more of the other devices may be wirelessly communicated (196) to one or more of: the devices 100a-100e; client device 999; resource 199; server 1160, AP 1170; NAS 1167; and data storage (1161, 1163), for example. As one example, user 800 may don a data capable wireless strapband 1120 positioned 1111 on a wrist body portion of user 800's left arm. Motion signals and/or biometric signals from other device 1120 may be wirelessly communicated 196 as described above and may be used in conjunction with other sensor signals and data to determine the state (I/C/N) of user 800 as described herein (e.g., as part of flow 1000 of FIG. 10).

User 800, client device(s) 999, and devices 100a-100e may be located in an environment that is remote from other elements of system 1100 such as resource 199, AP 1170, server 1160, data storage 1163, data storage 1161, NAS 1167, etc., as denoted by 1199. Wireless communication link 196 may be used for data communications between one or more of the elements of system 1100 when those elements are remotely located. One of the devices 100a-100e may be designated as a master device and the remaining devices may be designated as slave devices or subordinate devices as was described above. In some examples, regardless of a master/slave designation for the devices 100a-100e, the client device 999 may oversee, control, command, wirelessly (196) gather telemetry from sensor systems 340 of the devices 100a-100e, wirelessly query the devices 100a-100e, and perform other functions associated with devices 100a-100e (e.g., using APP 998).

As was described above in reference to flow 1000, first and second sensor data from one or more of the devices 100a-100e may be wirelessly (196) communicated to client device 999 as denoted by 1150. Client device 999 may perform processing and/or analysis of the sensor data or other data as denoted by 1151. Client device 999 may generate reports related to user 800's state (e.g., I/C/N) or other biometric, physiological, or psychological information concerning user 800's body, as denoted by 1153. Client device 999 may access data from one or more of the devices 100a-100e and/or other elements of system 1000, such as other device 1120, resource 199, server 1160, NAS 1167, or data storage (1161, 1163) as denoted by 1155. Client device 999 may process data and present coaching advice/suggestions as denoted by 1154, avoidance advice/suggestions as denoted by 1159, present notifications as denoted by 1152, and those data may be presented on a display of client device 999 or elsewhere, for example. Over Time as user 800's body changes and other environmental conditions that affect the user 800 change, client device 999 may calculate and set a baseline for a body part dimension D₀ and later as more Time has gone by, client device 999 may reset (e.g., re-calculate) the baseline, such that the baseline for D₀ may change over Time. In some examples, some or all of the functions performed by client device 999 may be performed by resource 198 (e.g., as denoted by 1150-1159), server 1160 or both.

Now, as was described above, determining the state (e.g., I/C/N) or the state of other biometric, physiological, or psychological information concerning user 800's body may not be instantaneously determinable and may in many cases be determinable over time. In FIG. 11, a temporal line for Time, another line for associated Activity of user 800, and a dashed line for Sampling of sensor data/signals and other data as described herein may be depictions of an ongoing process that continues and/or repeats over Time at a plurality of different intervals for the Time, Activity, and Sampling as denoted by t₀-tₙ for Time, a₀-aₙ for Activity, and s₀-sₙ for Sampling. One or more of the Activity and/or Sampling may continuously cycle 1177 over Time such that data from sensors and activity may be gathered, analyzed, and acted on by one or more elements of system 1100. As one example, a baseline value for dimension D₀ may change over Time as the activities of user 800 change and/or as changes occur within the body of user 800, such that over Time data from Sampling and Activity may result in dimension D₀ being repeatedly set and reset at Time progresses as described above in reference to 1157.

Given that Activity and/or Sampling may continuously cycle 1177 over Time, first and second sensor data may be changing, dimension D₀ may be changing, and therefore the data for determining the state (I/C/N) of user 800 may also be changing. Therefore, devices 100 and associated systems, client devices, and other elements, such as those depicted in FIG. 11 for system 1100 may be configured to adapt (e.g., in real time or near real time) to dynamic changes to user 800's body (e.g., health, weight, biometric, physiological, or psychological data, body portion 101 dimensions, baseline dimension D₀, etc.) to determine when signals from sensors 110, including any processing to eliminate errors caused by motion or other factors, are indicative of inflammation, contraction, or nominal states.

For example, when user 800 is asleep, Activity may be at minimum and Sampling may occur less frequently. On the other hand, when the user 800 is swimming, Activity may be high and Sampling may occur more often than when the user is sleeping. As lack of sleep may manifest as inflammation of body tissues, while the user 800 sleeps, motion signals from sensor system 340 or other sensors may be of lower magnitude and/or frequency, such that little or no processing may be required to determine if signals from sensors 110 are indicative of inflammation caused by lack of sleep. When user 800 wakes up, one or more of reports 1153, notifications 1152, or coaching 1154 may be presented to user 800 informing user 800 (e.g., using client device 999) of the inflammation and optionally advising or suggesting to user 800 steps to take (e.g., in diet, behavior, activity, stress reduction, fitness, etc.) to remediate the inflammation.

As another example, if user 800 is not properly hydrating (e.g., taking in enough fluids such as water), then while sleeping, little or no processing may be required to determine if signals from sensors 110 are indicative of contraction potentially caused by dehydration. When user 800 wakes up, one or more of reports 1153, notifications 1152, or coaching 1154 may be presented to user 800 informing user 800 (e.g., using client device 999) of the inflammation and optionally advising or suggesting to user 800 steps to take (e.g., in diet, behavior, activity, stress reduction, drink more water before exercising/swimming, how much more water to drink, etc.) to remediate the contraction.

Conversely, while user 800 is swimming, motion signals from sensor system 340 or other sensors may be of higher magnitude and/or frequency than when user 800 is sleeping, such that additional processing may be required to determine if signals from sensors 110 are indicative of inflammation caused by over training, strained or injured muscles/tissues, etc. After the swimming is over, ongoing sampling and processing of sensor data may determine that inflammation has been detected and the user 800 may be informed (e.g., using client device 999) via reports, notifications, etc., of the inflammation and optionally advising or suggesting to user 800 steps to take (e.g., in workout routine) to remediate the inflammation.

In FIG. 11 devices 100a-100e and 1120 may be configured to sense different activity in body of user 800 and may wirelessly communicate 196 data from their respective sensors, such as 100a being configured to sense fatigue, TRHR, I/C/N, and accelerometry (ACCL), 1120 configured to sense ACCL, 100d configured to sense I/C/N, TRHR, and ACCL, 100e configured to sense Fatigue and ACCL, 100b configured to sense I/C/N and ACCL, and 100c configured to sense I/C/N, fatigue, and TRHR, for example. In some examples, devices 100a-100e and 1120 may be configured to sense more or fewer types of activity than depicted.

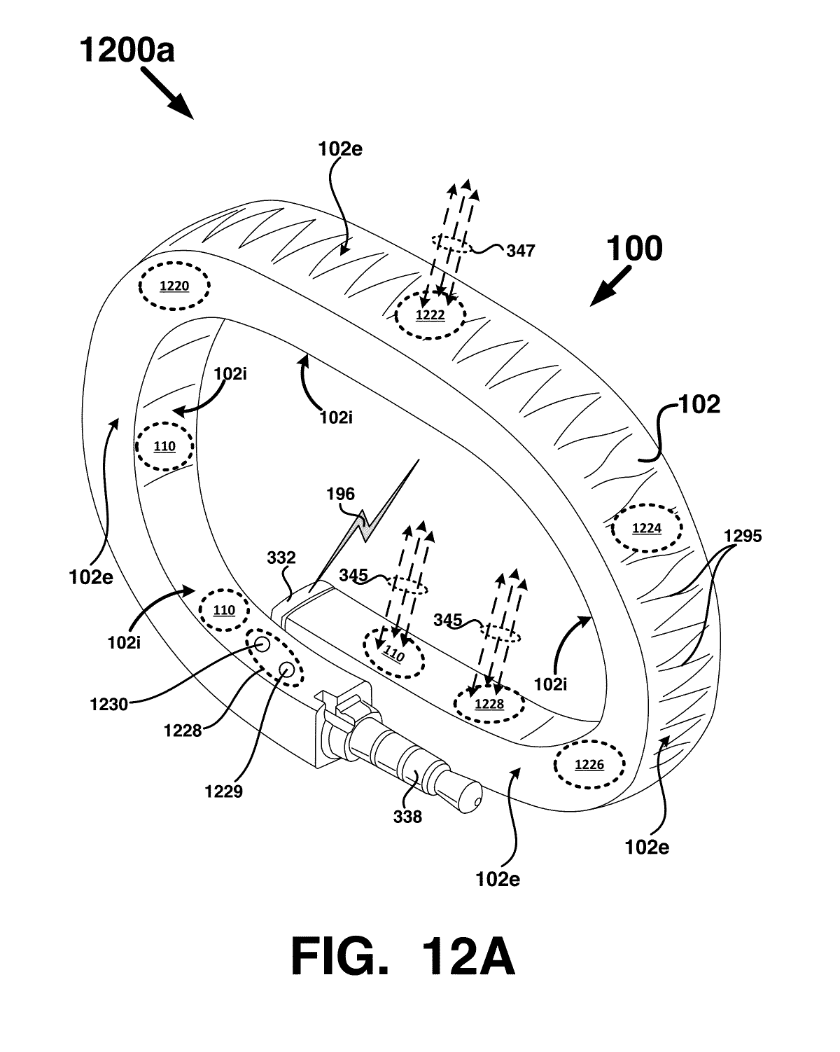

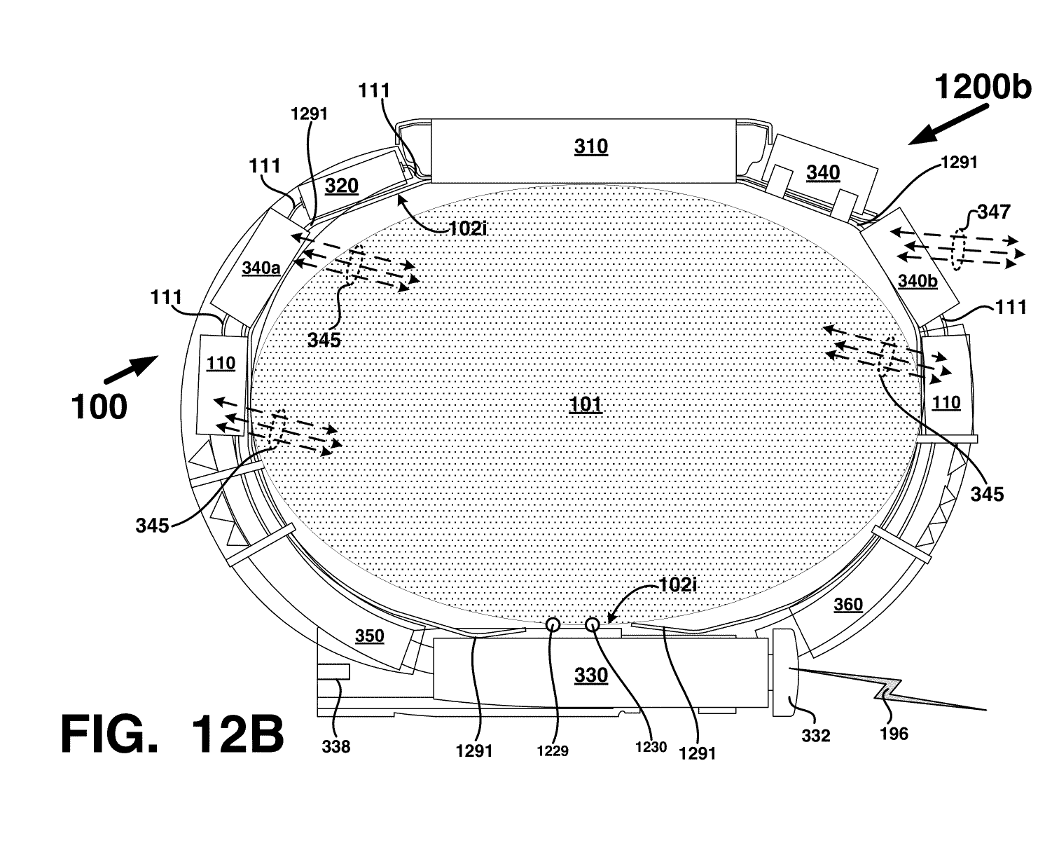

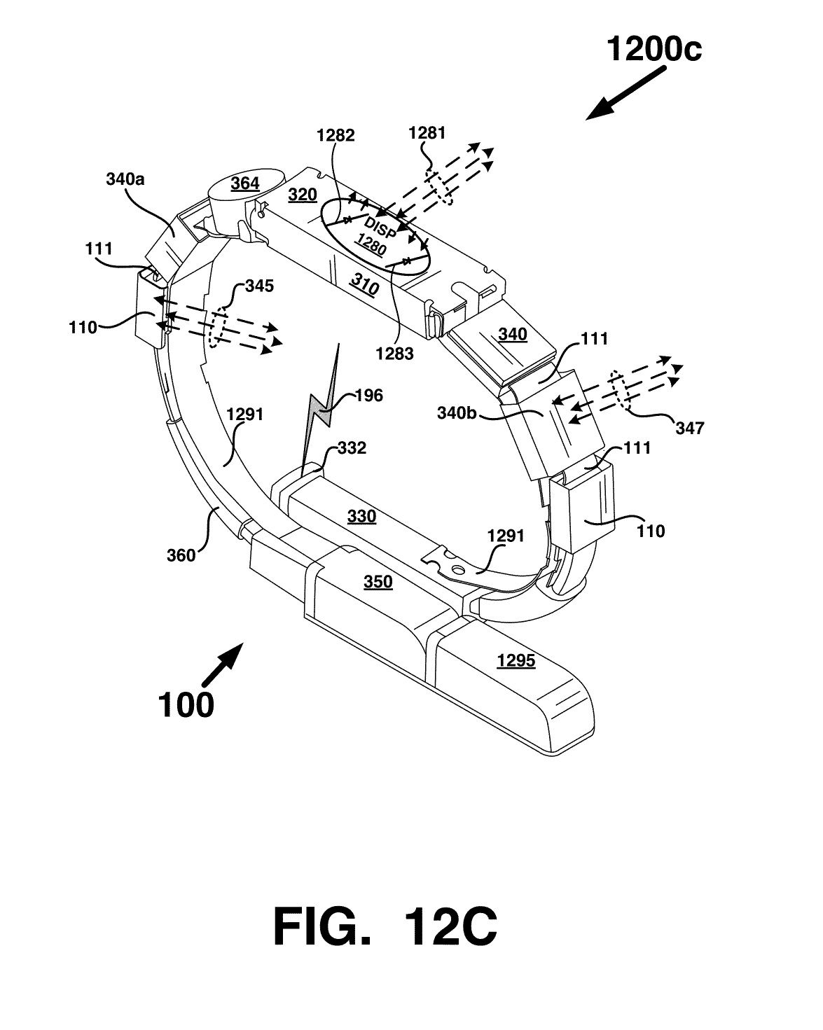

FIGS. 12A-12C depict different views of examples 1200a-1200c of a wearable device 100 to detect inflammation. In FIG. 12A, chassis 102 may comprise a flexible material and/or structure (e.g., a space frame, skeletal structure, spring or flat spring) configured to retain a shape once flexed or otherwise wrapped around or mounted to the body portion to be sensed by device 100 (e.g., the wrist, arm, ankle, neck, etc.). Exterior portions of chassis 102 may include a covering 102e that may include ornamental and/or functional structures denoted as 1295, such as for an aesthetic purpose and/or to aid traction or gripping of the device 100 by a hand of the user. Components of device 100 as described above in FIGS. 1 and 3 may be positioned within chassis 102. A variety of sensors may be positioned at one or more locations in device 100. As one example, sensor(s) 110 may be positioned on the interior portion 102i so as to be positioned to couple with or contact with body portion 101 (see FIG. 12B) for sensing 345 force exerted by the body portion 101. Similarly, other sensors, such as those for sensing biometric or other data from user 800's body may also be positioned to sense 345 the body portion 101, such as sensor 1228. For example, sensor 1228 may include one or more electrodes (1229, 1230) configured to contact tissue (e.g., the skin) of body portion 101 and sense electrical activity of the sympathetic nervous system (SNS) (e.g., arousal) on the surface of body portion 101, below the surface or both (e.g., dermal or sub-dermal sensing). Sensor 1228 and electrodes (1229, 1230) may be configured for sensing one or more of GSR, EMG, bioimpedance (BIOP) or other activity related to arousal and/or the SNS. Optionally, other sensors may be positioned in device 100 to sense 347 external events, such as sensor 1222 (e.g., to sense external temperature, sound, light, atmosphere (smog, pollution, toxins, cigarette smoke, chemical outgassing) etc.), or sensors 1220, 1224, 1226 for sensing motion. Device 100 may include a wired communication link/interface 338 such as a TRS or TRRS plug or some other form of link including but not limited to USB, Ethernet, FireWire, Lightning, RS-232, or others. Device 100 may include one or more antennas 332 for wireless communication 196 as described above.

In a cross-sectional view of FIG. 12B, an example positioning of components/systems of device 100 is depicted. Here, a substructure 1291, such as the aforementioned space frame, skeletal structure, spring or flat spring, may be connected with components or systems including but not limited to processor 310, data storage 320, sensors 110, communications interface 310, sensor system 340, 340a, 340b, I/O 360, and power system 350. Bus 111 or bus 301 may be routed around components/systems of device 100 and be electrically coupled with those components/systems. Some systems such as sensor system 340 may be distributed into different sections such as 340a and 340b, with sensors in 340a sensing 345 internal activities in body portion 110 and sensor 340b sensing 347 external activities. Port 338 is depicted as being recessed and may be a female USB port, lightning port, or other, for example. Port 338 may be used for wired communications and/or supplying power to power system 350, to charge battery 355, for example. Body portion 101 may be positioned within the interior 102i of chassis 102.

FIG. 12C depicts a profile view of another example positioning of internal components of device 100. An optional cap 1295 may be coupled with chassis 102 and may protect port 338 from damage or contamination when not need for charging or wired communications, for example. A transducer 364, such as a speaker and/or vibration motor or engine may be included in device 100. Notifications, reports, or coaching may be audibly communicated (e.g., speech, voice or sound) to user 800 using transducer 364. Device 100 may include a display, graphical user interface, and/or indicator light(s) (e.g., LED, LED's, RGB LED's, etc.) denoted as DISP 1280 which may be used to indicate a user's mood based on indications (I/C/N) and optionally other biometric data and/or environmental data as described above. The display and/or indicator lights may coincide with and/or provide notice of the above mentioned notifications, reports, or coaching. DISP 1280 may transmit light (e.g., for mood indication) or receive light (e.g., for ambient light detection/sensing via a photo diode, PIN diode, or other optoelectronic device) as denoted by 1281. Chassis 102 may include an optically transparent/translucent aperture or window through which the light 1281 may pass for viewing by the user 800 or to receive ambient light from ENV 198. As one example, one or more LED's 1282 may transmit light indicative of mood, as indications of (I/C/N), or other data. As another example, a photo-sensitive device 1283 may receive external light and generate a signal responsive to or indicative of an intensity of the light.

Referring now to FIG. 13 where a block diagram of an example 1300 of a cycle 1301-1306 of monitoring a user 800 having a wearable device to detect inflammation 100 and data inputs that may be used in a calculus for determining whether or not inflammation, contraction, or nominal states are indicated in the user 800 is depicted. There may be more or fewer data inputs than depicted in example 1300 as denoted by 1393. As time 1320 progresses, device 100 may receive, analyze, and process sensed signals generated by sensor system 340 as denoted by the arrow 340. At appropriate intervals, device 100 may communicate information including but not limited to notifications, advise, coaching, visual stimulus, audible stimulus, mechanical stimulus, user biometric data, data from sensor system 340, motion signal data, data from sensors 110, mood of user 800, almanac data, historical data, or any combination of the foregoing as denoted by arrow 1399.

Device 100 may receive information depicted in FIG. 13 and/or elsewhere herein from sources, systems, data stores, wireless devices, and devices, including but not limited to resource 199, client device 999, other wireless systems (e.g., via 196), from other devices 100, from other wireless devices such as exercise equipment, data capable strap bands, fitness monitors, smart watches or the like, reports, notifications, avoidance, coaching (RNC), compute engines (e.g., server 960 or computer system 200), biometric data, almanac data, historical data, or any combination of the foregoing as denoted by arrow 1398 adjacent to device 100.

In FIG. 13, one or more devices 100 may be included in an ecosystem 1310 of devices to measure inflammation or other health metrics (e.g., fatigue, resting heart rate) as denoted by 1390. User 800 may wear device 100i as a ring (e.g., see 600g in FIG. 6G) about a finger and the communication of information denoted by arrows 340, 1399, and 1398 as described above may apply to one or more of the wearable devices to detect inflammation and/or the other health metrics (e.g., such as 100, 100i) in ecosystem 1310. For example, device 100 may communicate 196 data from its sensor system 340 to device 100i, or vice-versa. As for the aforementioned three states of nominal (e.g., what is normal for user 800), inflammation, and contraction, over time 1320, dimension D of body portion 101 may vary in dimension from any of the aforementioned three states. Accordingly, over time 1320, dimension D of body portion 101 may cycle between any of D₀, Dᵢ, and D_{c} as one or more of the items of data, activities, environment, events, sensor signals, sensor data, etc., depicted outside of the dashed line for ecosystem 1310 affect user 800 and manifest in the body of user 800 as one of the three states.

Accordingly, starting clockwise at D₀, dashed line 1301 depicts body portion 101 transitioning from nominal to contraction D_{C}, dashed line 1303 depicts body portion 101 transitioning from contraction to inflammation Dᵢ, and dashed line 1305 depicts body portion 101 transitioning from inflammation to nominal D₀. Similarly, again using D₀ as a starting point and going in a counter-clockwise direction, dashed line 1302 depicts body portion 101 transitioning from nominal to inflammation Dᵢ, dashed line 1304 depicts body portion 101 transitioning from inflammation to contraction D_{C}, and dashed line 1306 depicts body portion 101 transitioning from contraction to nominal D₀. Therefore, over time 1320 the variations in dimension D of body portion 101b may change and may transition to/from any of the three states (I/C/N), and device 100 may be configured to monitor those changes and take necessary actions with respect to those changes at any desired interval such as constant (e.g., 24/7), at a less frequent interval (e.g., every ten minutes, every hour, eight times a day, etc.), or in response to a change in one or more of the items of data, environment, events, etc., that are depicted outside of the dashed line for ecosystem 1310 that may affect user 800 and may trigger monitoring by one or more of the devices 100. Although indications of the three states (I/C/N) may be monitored 24/7 or at some other interval, other biometric parameters (e.g., true resting heart rate), physiological state and/or psychological state (e.g., user fatigue) may be monitored as well, may be monitored in real time, and may be automatic with the user 800 being passive in his/her actions with respect to monitoring by device 100.

As discussed above, there are a plurality of items of data, environment, events, etc., that are depicted outside of the dashed line for ecosystem 1310 and there may be more or less than depicted as denoted by 1393 and the depictions in FIG. 13 may be a non-exhaustive list and comprise non-limiting examples presented for purposes of explanation only. For purposes of clarity these examples will be referred to collectively as datum. The datum may affect one or more of user 800's mental state, physical state, or both. Some of the datum may affect other datum, such work 1333 may impact stress 1343, for example. Or exercise 1338 may affect one or more types of biometric data 1378, for example. As another example, resting heart rate (RHR) 1375 may be affected by whether or not the user 800 is at sleep 1342, is at rest 1376, is under stress 1343, or is in a state of relaxation 1355. Some of the datum's may be data sensed by, collected by, processed by, or analyzed by one or more of the devices 100 or some other device. Some of the datum's may comprise specific data about user 800 and that data may or may not be static, and may include but is not limited to weight and/or percent body fat 1362, health data 1341 (e.g., from health history or health records), family 1335 (e.g., married, single, children, siblings, parents, etc.). Some of the datum's may be analyzed in context with other datum's, such as food/drink 1351, sugar 1363, or diet 1340 being analyzed in conjunction with location data 1360 which may be provided by an internal system of devices 100 and/or an external device (e.g., client device 999 or resource 199). For example, if user 800 experience inflammation (e.g., as reported by device 100 and/or 100i) due to a high sugar dosage from drinking a chocolate milk shake at an ice cream shop, location data may include a coffee shop (e.g., from eateries data 1350) the user 800 may be notified of via the notice function or coached to go to using the coaching function. The user 800 may be informed that caffeine may serve as an anti-inflammatory and to have a cup of coffee, latte, low or no sugar energy drink or other caffeinated drink/beverage to reduce the inflammation or return the user 800 to the nominal state. Location data may include history data from locations user 800 frequents, such as the ice cream shop, the coffee shop, grocery stores, restaurants, etc., just to name a few, for example. The reporting, notification, and coaching functions may again be invoked to inform the user 800 that his/her taking the prescribed action has either reduce the inflammation or returned the user's state to nominal.

Device 100i may indicate a mood of the user 800 using indicator lights 1282 (e.g., LED's) (e.g., see also 560 and 562 in FIG. 5) with only two of the five lights activated when the user 800 is experiencing the inflammation state due to the high sugar does and those two indicator lights 1282 may be indicative of the user 800 being in a sluggish or lethargic low energy mood due to insulin production in the user's body resulting from the high sugar dose. Conversely, after receiving the notification and/or coaching and taking affirmative action to remedy the inflammation by drinking the caffeinated beverage, four of the five indicator lights 1282 may activate to indicate reduced inflammation or a return to the nominal state. Those four indicator lights 1282 may be indicative of the user 800 being in a good mood (e.g., more energy). In some example, the reporting function may comprise using the indicator lights 1282 to report some change in body function or other information to user 800.

One or more of the reporting, notification, avoidance, coaching (RNC) may be presented on a display of client device 999 (e.g., using a GUI or the like) in a format that may be determined by APP 998, or other algorithms. Other systems of client device 999 may be used for RNC, such as a vibration engine/motor, ringtones, alarms, audio tones, music or other type of media, etc. As one example a song or excerpt from a song or other media may be played back when inflammation is detected and another song for when contraction (e.g., dehydration to extreme dehydration are indicated).

During the cycles depicted in FIG. 13, one or more of the datum's may be updated and/or revised as new data replaces prior data, such as the case for changes in the user 800's weight or body fat percentage 1362, diet 1340, exercise 1338, etc. The user 800 may input change in weight or body fat percentage 1362 using client device 999 (e.g., via the GUI and/or APP 998), or the user may use a wirelessly linked scale that interfaces (e.g., wirelessly) with device 100, device 100i, or client device 999 and updates the weight/% body fat. The cycles depicted in FIG. 13 may run (e.g., be active on one or more devices 100) on a 24/7 basis as described above and updates, revisions, and replacing prior data with new data may also occur on a 24/7 basis.

In FIG. 13 many non-limiting examples of information related to user 800 or havening an effect on user 800 are depicted to illustrate how numerous and broad the information that may be used directly, indirectly, or produced directly or indirectly by one or more devices 100. The following non-limiting examples of information may include but are not limited to: internal data 1331 may include any form of data used and/or produced internally in device 100 and internal data 1331 may be a superset of other data in device 100 and/or depicted in FIG. 13; external data 1332 may include any form of data used and/or produced external to device 100 and may be a superset of other data depicted in FIG. 13; work 1333 may be information related to work the user 800 does or a profession of user 800; school 1334 may be information relating to user 800's education, current educational circumstances, schooling of user 800's children; family 1335 may relate to user 800's immediate and/or extended family and relatives; friends 1335 may relate to friends of user 800; relationships 1337 may relate to intimate and/or societal relationships of user 800; weight and/or percent body fat 1362 may comprise actual data on those metrics and/or user goals for those metrics; circumstances 1361 may comprise events past, present or both that may affect or are currently affecting user 800; athletics 1339 may be data regarding athletic pursuits of user 800; biometric 1378 may comprise data from one or more devices 100, data from medical records, real-time biometric data, etc.; location 1360 may comprise data relating to a current location of user 800, past locations visited by user 800, GPS data, etc.; exercise 1338 may comprise information regarding exercise activity of user 800, exercise logs, motion and/or accelerometer data associated with specific exercises and/or exercise routines; health data 1341 may be any information from any source regarding health of user 800, such as medical records, etc.; diet 1340 may be information on a diet regime of user 800, dietary instructions for user 800, nutrition requirements for a diet (e.g., calories, carbohydrates, food quantizes), etc.; stress 1343 may be actual stress in user 800 as passively determined by device(s) 100, historical data on stress or stressful situations related to user 800, etc.; sugar 1363 may comprise data one sugar intake by user 800 or sensor data indicating an effect of sugar on user 800, or locations (e.g., from location 1360) determined to be associated with high sugar intake by user 800 (e.g., an ice cream shop the user patronizes); at rest 1376 may include any data related to user 800 when the user is at rest and is not sleeping such as biometric data, respiration, arousal, HR, TRHR, HRV, accelerometry, etc.; sleep 1342 may include any data related to user 800 when the user is sleeping such as time of sleep, quality of sleep, respiration, arousal, biometric data, HR, TRHR, HRV, accelerometry, etc.; status 1359 may include data about user 800's social, professional, economic, or financial status as status may have bearing on the emotional and/or physical state of user 800; inactivity 1346 may include data on periods and/or patterns of inactivity of user 800 and sensor data associated with the inactivity such as accelerometry, arousal, HR, HRV, TRHR, arousal, and other biometric data, where inactivity may be one indicator of fatigue and/or depression; travel 1347 may include any data related to how travel may affect user 800 such as stress, fatigue, HR, HRV, arousal, biometric data, diet, sleep, I/C/N, etc., travel 1347 may be combined with other data such as location data 1360 to determine if travel to/from certain destinations have a positive or negative physical and/or mental impact on user 800; commute 1344 may include any data related to how commuting may affect user 800 such as stress, fatigue, HR, HRV, arousal, biometric data, diet, sleep, I/C/N, etc., travel 1347 may be combined with other data such as location data 1360 or travel 1347 to determine if commuting to/from certain destinations, commute distances, commute times, etc., have a positive or negative physical and/or mental impact on user 800; RESP 1345 may include any data related to respiration of user 800 such as at rest, while sleeping, when under stress, when fatigued, when dehydrate or suffering inflammation (I/C/N), during exercise or other forms of physical exertion, mental exertion, etc.; depression 1352 may include any data related to depression in user 800 include mental or health records, past incidents of detected depression, fatigue, stress, accelerometry, arousal, biometric data, etc.; news 1357 may include any data related to news from a media source or other that may positively or negatively affect user 800 and news 1357 may be received on an external device such as client device 999 and APP 998 may be configured to parse news of interest to user 800 and push data for relevant news (e.g., affects user 800) to device 100; mood 1353 may include any data relating to a mood (e.g., physical and/or mental) of user 800 such as feeling up, down, depressed, fatigued, stressed, or one of the moods indicated by indicators (1282, 561) of devices 100i or 100; finances 1356 may include any data related to financial status or circumstances related to user 800 as financial conditions may have an effect on the metal and/or physical state of user 800; weather 1350 (e.g., weather conditions) may affect user 800's mind 800m and/or body 1350 and may include any data including data from web sites, other locations, or sources that monitor or forecast weather and weather 1350 may be used in conjunction with location 1360 to determine weather conditions proximate the user 800's current location, and weather 1350 may include historical data (e.g., collected over time) on how weather affects user 800; caffeine 1349 may include data on locations (e.g., from location 1360) where user 800 obtains food and/or drink containing, conditions under which user 800 resorts to taking caffeine, and amount of caffeine intake/consumption by user 800; eateries 1350 may include locations (e.g., from location 1360) where user 800 obtains nourishment, has meals, has snacks, has food/drink, etc. and location 1360) may be used to determine the types of food/drink associated with the eateries and that information may be used to determine diet information, compliance with a diet plan, for advice or counseling about diet, etc.; food/drink 1351 may include data on types and quantities of food and/or drink the user 800 has consumed and food/drink 1351 may be related to or used in conjunction with other data such as eateries 1350, caffeine 1349, sugar 1363, diet 1340, location 1360, or others; GAIT 1381 may include data regarding motion and/or accelerometry of user 800 including movement of limbs, speed of movement, patterns, duration of activity that generated data included in GAIT 1381, and history of previous GAIT data that may be compared against current and/or real-time gate data; seasons 1358 may be any data related to the seasons of the year and how those seasons affect user 800, seasons 1358 may be tied or otherwise used in conjunction with weather 1350; ACCL (accelerometry) 1379 may include any data (e.g., motion sensor signals) related to movement of user 800's body and may include real-time and/or historical data on accelerometry of user 800 under different conditions/activities, ACCL 1379 may include data that may be used to determine if motion of user 800 is too low (e.g., user 800 may be fatigued) or too high (e.g., user 800 is stressed or anxious); injury 1348 may include any data relating to a current injury or history of past injuries to user 800 and may include data from other items such as health data 1341; disease 1354 may include any data relating to a current disease or history of past diseases for user 800 and may include data from other items such as health data 1341; relaxation 1355 may include any data related to activities associated with a relaxed state of user 800's mental state, physical state or both; arousal 1373 may include any data including historical data and sensor signals that relate to muscle and/or electrical activity in the sympathetic nervous system (SNS) of user 800; SNS (sympathetic nervous system) 1372 may include any data including historical data and sensor signals (e.g., GSR, EMG) that relate to muscle and/or electrical activity in the sympathetic nervous system (SNS) of user 800 and may be similar to arousal 1373 and may include the same or different data than arousal 1373; HR (heart rate) 1383 may be any data including sensor signals related to heartbeat (e.g., in bpm) of user 800 and may include historical data on heartbeat of user 800; HRV (heart rate variability) 1383 may be any data including sensor signals related to HRV of user 800 and may include historical data on HRV of user 800; TRHR (true resting heart rate) 1375 may include any data, history, real-time data, or other forms of information related to the TRHR of user 800; temperature 1380 may include data about body temperature (e.g., in real-time) and/or historical body temperature of user 800; and almanac data 1377 may broadly include any data that may be accessed by device(s) 100 or external devices that may be used in processing, calculating, analyzing, coaching, avoidance, reporting, notifications, advising, or the like and may include data generated by one or more systems of device(s) 100 such as the sensor system 340 or others.

One or more of the items of information/data described in the foregoing examples for FIG. 13 may be used for passively determining (e.g., in real-time) stress, fatigue, inflammation, contraction, nominal states (I/C/N), arousal of the SNS, true resting heart rate (TRHR), or other data that may be gleamed from user 800 using the systems of device(s) 100, etc. as described herein. Data in some of the items of data may be duplicated and/or identical to data in other of the items of data. Device(s) 100 and/or external systems (e.g., 199 or 999) may update, revise, overwrite, add, or delete data from one or more of the items depicted in FIG. 13. As one or more of the devices 100 operate continuously (e.g., 24/7), on an intermittent basis or both, data in one or more of the items may be changed by new data from one or more of the devices 100. Some of the devices 100 may access different sub-sets of the items such as devices 100 with only biometric sensor may not write data to ACCL 1379 but may read data from ACCL 1379; whereas, a device 100 having motion sensors may write sensor data to ACCL 1379 and may optionally read data from ACCL 1379 (e.g., motion signal data from other wirelessly linked devices 100) to perform analysis, calculations, etc. for example. Data in one or more items in FIG. 13 may be a source for data inputs (e.g., 1601-1617) depicted in FIG. 16 below or may derive from signals generated by sensors in sensor system 340 (e.g., in FIG. 16).

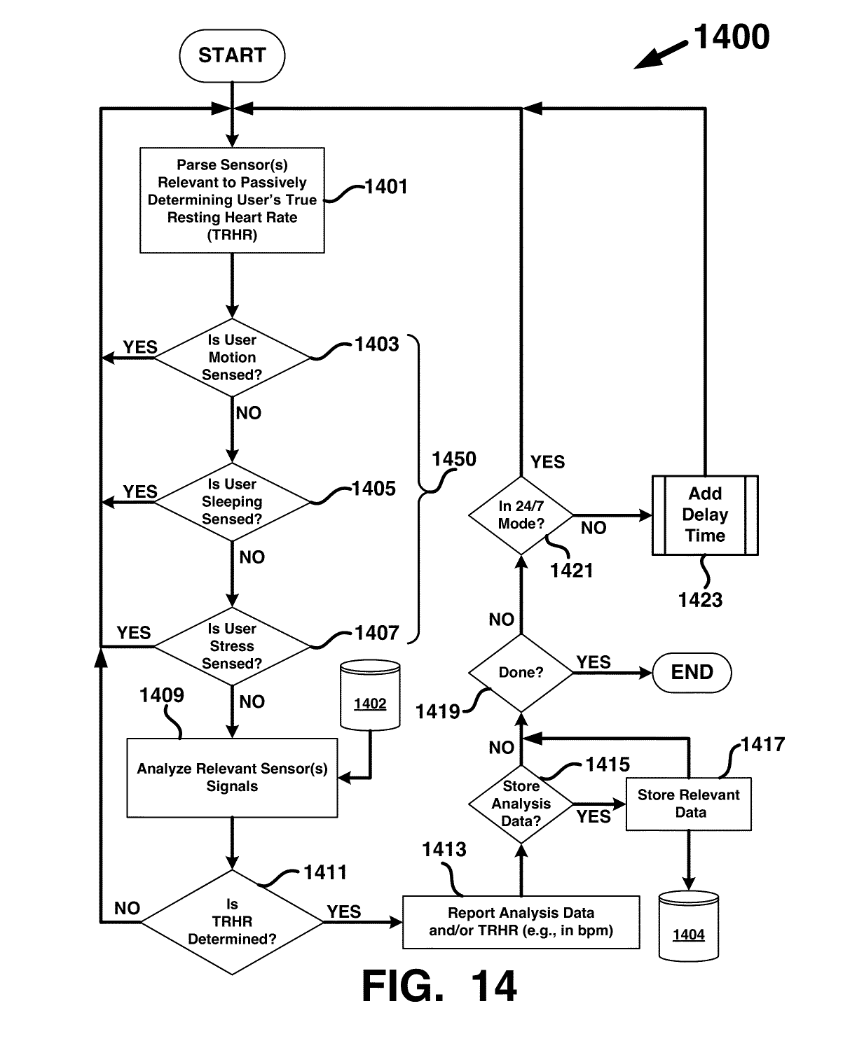

Attention is now directed to FIG. 14 where one example of a flow diagram 1400 for passively determining a true resting heart rate (TRHR) of a user 800 is depicted. At a stage 1401 sensors in sensor system 340 in device 100 or in another device 100 wirelessly linked with device 100 that are relevant to passively determining TRHR of user 800 may be parsed (e.g., scanned, interrogated, analyzed, queried, receive, read, or activated). Relevant sensors may comprise all or a sub-set of sensors in sensor system 340 of device 100 and/or another device 100 that generate signals that may be processed, analyzed, or otherwise applied to determine the TRHR. Relevant sensors may comprise selected sensors in sensor system 340 of device 100 and/or another device 100 that generate signals that may be processed, analyzed, or otherwise applied to determine the TRHR. Passively may comprise the user 800 doing nothing at all (e.g., taking no action) to assist or otherwise make the determination of TRHR happen. In some examples, user 800 may instruct device(s) 100 (e.g., via the APP on client device 999) to activate one or more modes of operation, such as the TRHR mode, the I/C/N mode as described above, or a fatigue mode, as will be described below. To that end, the only action on behalf of the user 800 may be to activate the TRHR mode. In some examples, the TRHR mode and/or determining TRHR may be automatically active on device(s) 100 (e.g., at power up) and the user 800 is passive as to its operation. Similarly, the I/C/N and fatigue determinations and/or modes may also be automatic and the user 800 is passive as to their operation.

At a stage 1403 signals from one or more sensors and/or sensor types for sensing motion may be analyzed to determine whether or not the user 800 is in motion. An indication of motion (e.g., above a threshold value in G's or G's unit of time G's/sec) may mean the user 800 is not at rest. If a YES determination is made, then flow 1400 may transition to another stage, such as cycling back to the stage 1401, for example. TRHR may comprise a state of user 800 in which the user 800 is at rest (e.g., low or no accelerometry (motion signals attributed to human movement) in user 800), is not asleep, and is not stressed (e.g., physically and/or mentally). Here, a YES determination of motion being sensed (e.g., via motion sensors in 340) may indicate that the user 800 is not at rest and one or more biometric signals such as heart rate (HR), heart rate variability (HRV), or arousal activity in the sympathetic nervous system (SNS) may not be reliably used in a determination of TRHR, until such a time as the NO branch is may be taken from the stage 1403. At rest may comprise the user 800 being awake (e.g., not sleeping) and not in motion, where not in motion may not mean absolutely still, but rather not exercising, not walking, not talking, etc. For example, at rest may comprise the user being awake and lying down on a sofa, sitting on a chair, or riding on a train?

If the NO branch is taken, then flow 1400 may transition to a stage 1405 where a determination may be made as to whether or not one signals from sensors in 340 indicate that the user 800 is asleep. Motion signals (e.g., from an accelerometer and/or gyroscope) and other signals such as biometric signals from HR sensors, HRV sensors, SNS sensors (e.g., GSR, EMG, bioimpedance), respiration sensors (RES), or others, may be used singly or in combination to determine if the user 800 is sleeping. If a YES branch is taken, then flow 1400 may transition to another stage, such as cycling back to the stage 1401, for example. If a NO branch is taken, then flow 1400 may transition to a stage 1407 where signals from one or more sensors in 340 may be analyzed to determine if the user 800 is stressed. Motion signals (e.g., from an accelerometer and/or gyroscope) and other signals such as biometric signals from HR sensors, HRV sensors, SNS sensors (e.g., GSR, EMG, bioimpedance), respiration sensors (RES), I/C/N sensors 110, or others, may be used singly or in combination to determine if the user 800 is stressed. Stress may comprise mental state (e.g., arousal in the SNS), emotional state (e.g., angry, depressed), physical state (e.g., illness, injury, inflammation, dehydration), metal activity (e.g., solving a difficult problem), of some combination of those (e.g., fatigue) for example. If a YES branch is taken, then flow 1400 may transition to another stage, such as cycling back to the stage 1401, for example. If a NO branch is taken, the flow 1400 may transition to a stage 1409. A taking of the YES branch from one or more of the stages 1403-1407 which are denoted as group 1450 may comprise continually parsing the relevant sensors (e.g., in sensor system 340) until analysis of signals from the relevant parsed sensors allows each NO branch in group 1450 to be taken so that flow 1400 arrives at the stage 1409. For example, sensor signals indicating the user 800 is at rest, is not asleep, and is not stressed may allow entry into the stage 1409.

At the stage 1409, sensor signals that are relevant to a passive determination of TRHR are analyzed (e.g., using processor 310). Passive determination, as described above, does not require any action on part of user 800. Analysis at the stage 1409 may include using one or more sensors in 340 to determine the user 800's HR and/or HRV while the conditions precedent to entry into the stage 1409 are still present, that is the NO branches of group 1450 are still valid (e.g., user 800 is at rest, is not asleep, and is not stressed). Data 1402 may be used as an input for the analysis at the stage 1409. Data 1402 may include but is not limited to normal values of HR, HRV, GSR, RES, EMG, BIOP, or other measured norms for user 800. Data 1402 may include prior determined values of TRHR for user 800, for example. Data 1402 may include one or more of the datum's described above in reference to FIG. 13.

At a stage 1411, a decision may be made as to whether or not the analysis at the stage 1409 has determined TRHR (e.g., in bpm) for user 800. In a NO branch is taken, then flow 1400 may transition to another stage, such as cycling back to the stage 1401 where the stages in group 1450 may be repeated until all NO branches are taken to the stage 1409. The NO branch may be taken for a variety of reasons, such as conflicting sensor signals, for example. As one example, if HR is increasing and HRV is also increasing, then stage 1411 may determine that a TRHR value passively determined at the stage 1409 is inaccurate due to both HR and HRV increasing, where, typically as HR increases, HRV decreases. As another example, if GSR increases and HR decreases, then conflict in those signal readings may cause execution of the NO branch as HR typically increases with an increase in GSR. As yet another example, if GSR is indicative of low stress in user 800, but I/C/N indicates systemic inflammation, then there may be a conflict in those indicators because systemic inflammation typically affects arousal in the SNS and causes an increase in GSR. If a YES branch is taken, then TRHR has been determined and flow 1400 may transition to a stage 1413.

At the stage 1413, the TRHR may be reported (e.g., to a data store and/or display on client device 999 or other device) and/or analysis data (e.g., from stage 1409 and/or 1411) may be reported (e.g., to a data store and/or display on client device 999 or other device). An example of a data store may include but is not limited to a data storage system in resource 199, client device 999, one or more devices 100, DS 963, DS 961, the Cloud, the Internet, NAS, Flash memory, etc., just to name a few. In some examples, the stage 1413 may be optional and may not be executed in flow 1400.

At a stage 1415 a determination may be made as to whether or not to store the analysis data. If a YES branch is taken, then at a stage 1417 relevant analysis data (e.g., TRHR or other data from stage 1409 and/or 1411) is stored (e.g., in a data store 1404). Data store 1402 may include data that was stored at the stage 1417. One or more datum depicted in FIG. 13 may be revised and/or updated based on the analysis data. In some examples, data stores 1402 and 1404 may be the same data store. Subsequent to storing the data, flow 1400 may transition to a stage 1419, which is the same stage flow 1400 may transition to if the NO branch was taken from the stage 1415.

At the stage 1419 a determination may be made as to whether or not flow 1400 is Done (e.g., no more stages need to be executed). If a YES branch is taken, flow 1400 may terminate (e.g., END). If a NO branch is taken, flow 1400 may transition to a stage 1421.

At the stage 1421, a determination may be made as to whether or not a 24/7 mode is active (e.g., is set) on device(s) 100. If a YES branch is taken, then flow 1400 may transition to another stage, such as to the stage 1401 to begin again the parsing of relevant sensor(s) as was described above. The taking of the YES may repeat over and over again so long as the 24/7 mode is set (e.g., either by default or user 800 setting the mode), such that passively determining the TRHR of user 800 is an ongoing process that repeats and may update values of TRHR as appropriate over time as changes in the user's 800 physical and mental states change over time. In some examples, algorithms and/or hardware in device(s) 100 may clear the 24/7 mode so that the NO branch will be taken at the stage 1421. For example, if fatigue, inflammation, or dehydration are indicated, then device(s) 100 may clear the 24/7 mode and focus their processing, analysis, reporting, notifications, coaching, etc. on addressing those indications, and then at some later time the device(s) 100 may set the 24/7 mode so that the YES branch may be taken in future iterations of flow 1400.

If the NO branch is taken, then flow 1400 may transition to a stage 1423 where a time delay may be added to delay transition of flow 1400 back to the stage 1401. The time delay added may be in any time increment without limitation, such as sub-seconds, seconds, minutes, hours, days, weeks, etc.

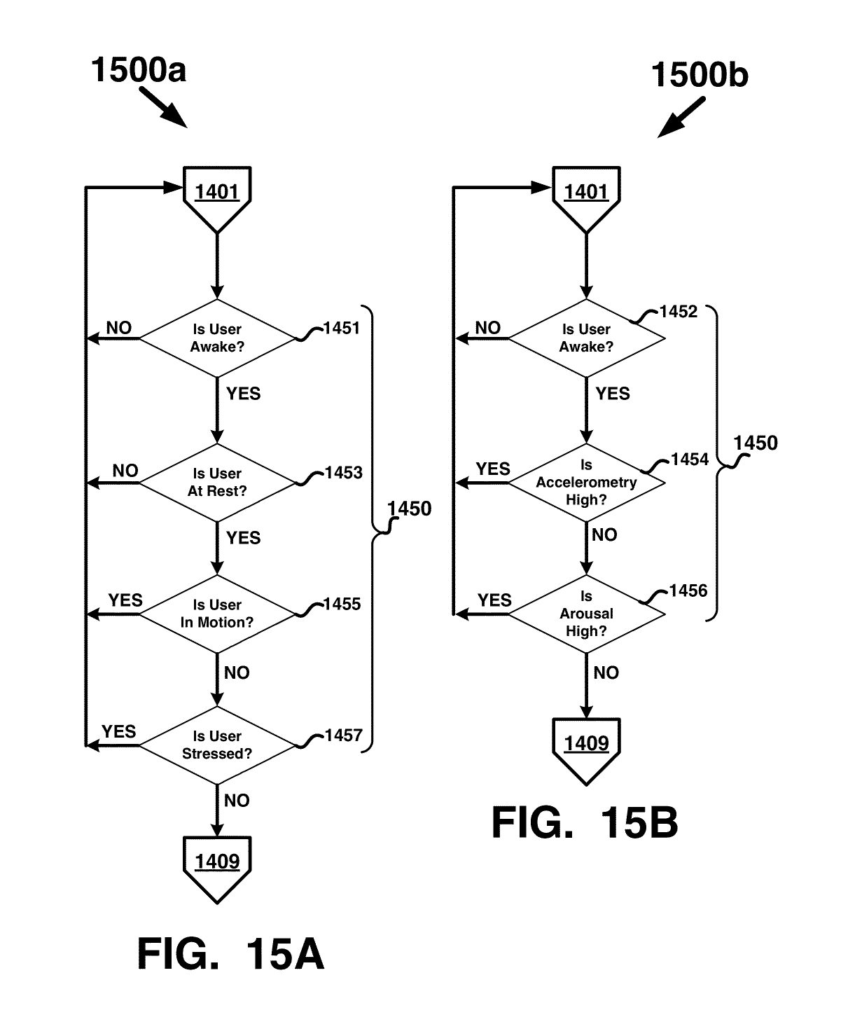

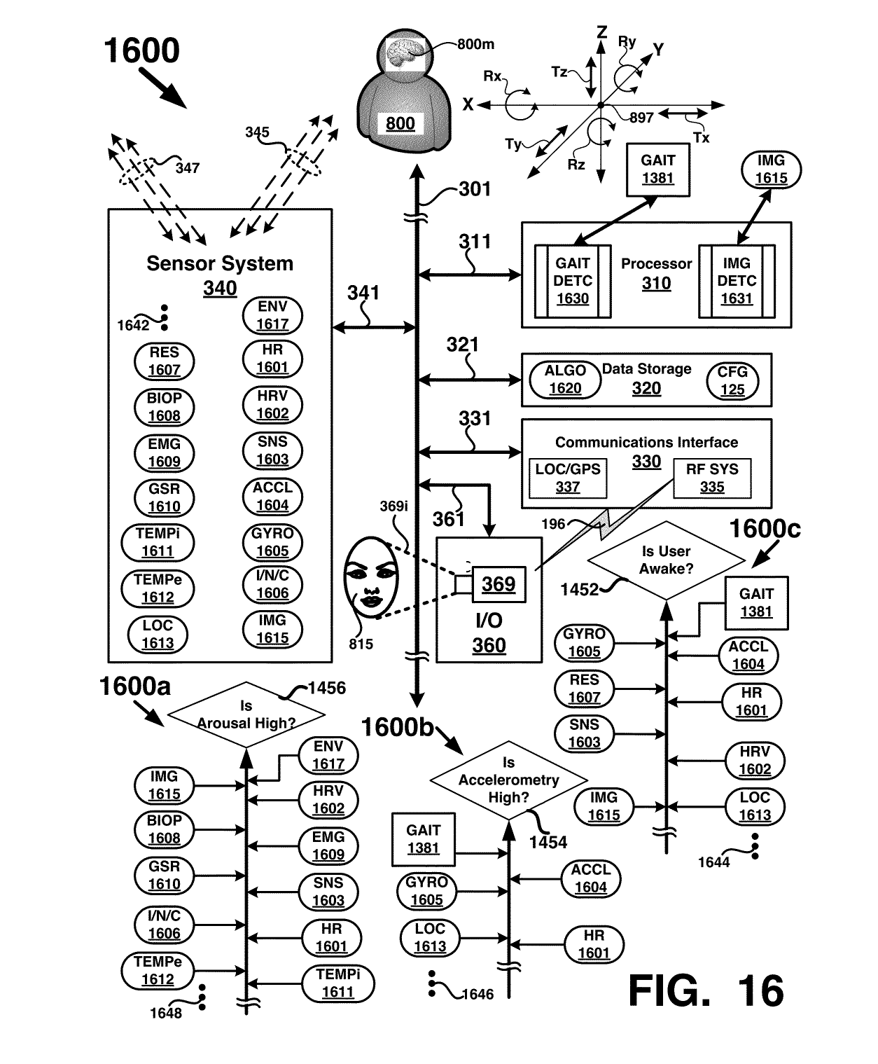

Reference is now made to FIGS. 15A-15B where two different examples (1500a, 1500b) of sensed data that may be relevant to passively determining TRHR of the user 800 are depicted. In FIG. 15A, group 1450 includes four determinations instead of the three (1403-1407) depicted in FIG. 14. Here, assuming entry from a prior stage, such as the stage 1401 of FIG. 14, at a stage 1451 one or more relevant sensor in 340 may be parsed to determine if the user 800 is awake (e.g., motion sensors and/or biometric sensors). At a stage 1453, one or more relevant sensor in 340 may be parsed to determine if the user 800 is at rest (e.g., motion sensors and/or biometric sensors). At a stage 1455, one or more relevant sensor in 340 may be parsed to determine if the user 800 is in motion (e.g., motion sensors, GAIT detection, biometric sensors). At a stage 1457, one or more relevant sensor in 340 may be parsed to determine if the user 800 is stressed (e.g., biometric sensors, HR, HRV, GSR, BIOP, SNS, EMG). Successful execution of stages 1451-1453 (e.g., branches taking YES, YES, NO, NO) may transition the flow of example 1500a to another stage, such as the stage 1409 of FIG. 14.

In FIG. 15B, group 1450 includes three determinations that may be different than the three (1403-1407) depicted in FIG. 14. Here, assuming entry from a prior stage, such as the stage 1401 of FIG. 14, at a stage 1452 one or more relevant sensor in 340 may be parsed to determine if the user 800 is awake (e.g., motion sensors and/or biometric sensors). At a stage 1454, one or more relevant sensor in 340 may be parsed to determine if accelerometry of the user 800 is high (e.g., motion sensors, GAIT detection, location data). At a stage 1456, one or more relevant sensor in 340 may be parsed to determine if arousal in the SNS of user 800 is high (e.g., GSR, BIOP, SNS, EMG, I/C/N). Successful execution of stages 1452-1456 (e.g., branches taking YES, NO, NO) may transition the flow of example 1500b to another stage, such as the stage 1409 of FIG. 14. High accelerometry and/or high arousal may be threshold values that exceed normal values of accelerometry and/or arousal in the user 800 (e.g., normal values for user 800 when awake, at rest and not aroused).

The determinations in examples 1500a and 1500b may ask similar questions but may parse different sets of sensors to select a YES or NO branch. For example, high accelerometry at the stage 1454 may forego parsing biometric sensors; whereas, stages 1453 and 1455 may parse biometric sensors to determine if the user 800 is at rest and in motion. Stage 1454 may include parsing of biometric sensors as motion by user 800 may affect HR, HRV, SNS, etc. However, high accelerometry may be determined without parsing biometric sensors. There are a variety of relevant sensors that may be parsed to passively determine TRHR, and the above groupings are non-limiting examples only. In some examples, the number and/or types of sensors that are parsed may be changed or altered during execution of flow 1400, of example 1500a, or of example 1500b. As one example, if a determination fails and flow returns to the stage 1401, a mix of sensors used for the next pass through group 1450 may change (e.g., biometric sensor are parsed for the stage 1454 or I/C/N is parsed for the stage 1457).

Description now turns to FIG. 16 where a block diagram 1600 of non-limiting examples of relevant sensor signals that may be parsed, read, scanned, and/or analyzed for passively determining a true resting heart rate (TRHR) of a user are depicted. Referring back to FIG. 3, sensor system 340 of device 100 may include a plurality of different types of sensors (e.g., force and/or pressure 110, motion, biometric, temperature, etc.) and signals from one or more of those sensors may be coupled (341, 301) with processor 310, data storage 320, communications interface 330, and other systems not depicted in FIG. 16. Communications interface 330 may transmit 196 via RF system 335 sensor signals from 340 and/or may receive 196 sensor signals via RF system 335 from one or more of other devices 100, external systems, and wireless client devices, for example. Sensor signals from 340 may be stored for future use, for use in algorithms executed internally on processor 310 and/or externally of device 100, may be stored as historical data, may be stored as one or more datum's depicted in FIG. 13, for example.

In sensor system 340, examples of sensors and their respective signals that may be relevant to determining TRHR and/or other states/conditions of user 800's physical and/or mental state (e.g., I/C/N, fatigue, mental state of user 800's mind 800m, etc.) include but are not limited to: sensor 1601 for sensing heart rate (HR); sensor 1602 for sensing heart rate variability (HRV); sensor 1603 for sensing activity (e.g., electrical signals) associated with the sympathetic nervous system (SNS) which may include activity associated with arousal; sensor 1604 for sensing motion and/or acceleration, such as a single-axis accelerometer or a multiple-axis accelerometer (ACCL); sensor 1605 for sensing motion and/or acceleration, such as one or more gyroscopes (GYRO); sensor 1606 for sensing inflammation, nominal, and contraction states of tissues of a user (e.g., sensor 110) (I/C/N); sensor 1607 for sensing respiration (RES); sensor 1608 for sensing bioimpedance (e.g., using sub-dermal current applied by electrodes) (BIOP); sensor 1609 for sensing electromyography (EMG); sensor 1610 for sensing skin conductivity, galvanic skin response, etc., at the dermal layer (GSR); sensor 1611 for sensing an internal temperature of user 800's body (TEMPI); sensor 1612 for sensing temperature external to user 800's body (e.g., ambient temperature) (TEMPe); sensor LOC 1613 for sensing location of user 800 via GPS or other hardware (e.g., client device 999) and/or software; and sensor IMG 1615 for image data (e.g., micro-expression detection/recognition, facial expression and/or posture recognition). IMG 1615 may be from image capture device 369 of FIG. 3, for example. IMG 1615 may be positioned in an external device (e.g., client device 999) and image data from IMG 1615 may be wirelessly transmitted to one or more devices 100 or to an external resource (e.g., 199, 960, 999) for processing/analysis, for example.

In some examples, device 100 or another device or system in communication with device 100 may sense an environment (e.g., 399) user 800 is in for environmental conditions that may affect the user 800, such as light, sound, noise pollution, atmosphere, etc. Sensors such as light sensors, ambient light sensors, acoustic transducers, microphones, atmosphere sensors, or the like may be used as inputs (e.g., via sensor signals, data, etc.) for sensor system 340 or other systems and/or algorithms in device 100 or a system processing data on behalf of one or more devices 100. ENV 1617 denotes one or more environmental sensors. More or fewer sensors may be included in sensor system 340 as denoted by 1642.

Some of the sensors in 340 may sense the same activity and/or signals in body of the user 800, such as EMG 1609, BIOP 1608, GSR 1610 which may be different ways of sensing activity in the sympathetic nervous system (SNS) and those sensors may be sub-types of SNS 1603. As another example, ACCL 1604 and GRYO 1605 may sense similar motion activity of user 800 as depicted by the X-Y-Z axes. GRYO 1605 may provide motion signals for rotation Rx, Ry, Rz about the X-Y-Z axes and ACCL 1604 may provide motion signals for translation Tx, Ty, Tz along the X-Y-Z axes, for example. In some examples, some of the sensors depicted may be determined by applying calculations and/or analysis on signals from one or more other sensors, such as sensing HR 1601 and calculating HRV from signal data from HR 1601. Signals from one or more sensors may be processed or otherwise analyzed to derive another signal or input used in determining TRHR, such as using motion signals from ACCL 1604 to determine a gait of user 800 (e.g., from walking and/or running). Those signals may be processed or otherwise analyzed by a gait detection algorithm GAIT DETC 1630, any output from GAIT DETC 1630 may be used in determinations of accelerometry 1454 and/or determinations of the user 800 being awake 1452, for example. GAIT DETC 1630 may output one or more signals and/or data denoted as GAIT 1381. GAIT 1381 may serve as an input to one or more stages of flow 1400, example 1500a, or 1500b. GAIT 1381 may comprise one of the datum's of FIG. 13 and may be used in present determinations (e.g., stage 1454, 1452 of FIG. 16) related to user 800 and/or future determinations (e.g., as historical data) related to user 800.

As one example of how signals from one or more sensors in 340 may be relevant to determining TRHR and/or relevant to one or more stages used for determining TRHR of user 800, the stage 1456, which determines if arousal is high (e.g., in user 800's sympathetic nervous system (SNS)), hardware and/or software may receive as inputs, signals from one or more relevant sensors including but not limited to: BIOP 1608; GSR 1610; SNS 1603; EMG 1609; ENV 1617; HR 1601; HRV 1602; I/N/C 1606; IMG 1615 (e.g., micro-expression on face 815 of user 800); TEMPi 1611; and TEMPe 1612.

As another example, determining of accelerometry is high at the stage 1454 may include one or more relevant sensors and their respective signals including but not limited to: ACCL 1604; GYRO 1605; LOC 1613; HR 1601; and GAIT 1381.

As yet another example, determining if the user 800 is awake at the stage 1452 may include one or more sensors and their respective signals including but not limited to: RES 1607; HR 1601; HRV 1602; SNS 1603; LOC 1613; GYRO 1605; ACCL 1604; IMG 1615 (e.g., process captured images for closed eyes, motion from rapid eye movement (REM) during REM sleep, micro-expressions, etc.); and GAIT 1381. In the examples above, there may be more of fewer sensors and their respective signals as denoted by 1648, 1646, and 1644. Some of the signals may be derived from signals from one or more other sensors including but not limited to HRV 1602 being derived from HR 1601, LOC 1613 being derived from LOC/GPS 337 signals and/or data, GAIT 1381 being derived from ACCL 1604, for example.

Processor 310 may execute one or more algorithms (ALGO) 1620 that may be accessed from data storage system 320 and/or an external source to process, analyze, perform calculations, or other on signals from sensors in 340 and/or signals or data from external sensors as described above. Some of the algorithms used by processor 310 may reside in CFG 125. APP 998 in client device 999 and/or applications, software, algorithms executing on external systems such as resource 199 and/or server 560 may process, analyze, and perform calculations or other on signals from sensors in 340 in one or more devices 100. As one example, accurate TRHR determinations may require indications that the user 800 is not experiencing physiological stress or other activity that may affect the mind 800m. Therefore, arousal related sensors and their respective signals (e.g., BIOP, EMG, GSR, SNS) and optionally other biometric signals (e.g., HR, HRV, RES, I/C/N), may be analyzed to determine if a state of the user 800's mind 800m is such that the user 800 is not stressed physiologically (e.g., the user 800 is in a peaceful state of mind and/or body). As another example, accelerometry of the user 800's body may be caused by motion of the user 800 and/or motion of another structure the user 800 is coupled with, such as a vehicle, an escalator, an elevator, etc. Therefore, sensor signals from LOC 1613, ACCL 1604 and/or GYRO 1605, GAIT 1381, may be processed along with one or more biometric signals (e.g., HR 1601, SNS 1603) to determine if accelerometry is due to ambulatory or other motion by the user 800 or to some moving frame of reference, such as a train, that the user 800 is riding in. Therefore, at the stage 1454, if GYRO 1605 and/or ACCL 1604 indicate some motion of user 800, GAIT 1381 is negligible (e.g., the user 800 is not walking), HR 1601 is consistent with a normal HR for the user 800 when awake and at rest, and LOC 1613 indicates the user 800 is moving at about 70 mph, then accelerometry may not be high and a determination of TRHR may proceed because a large component of the motion may be the train the user 800 is riding in, and motion of the user 800 may be due to slight movements made while sitting and/or swaying motion or others of the train. On the other hand, if the user 800 is slowly riding a bicycle, the movement of the user 800's legs, plus increase HR 1601, signals from GYRO 1605 and/or ACCL 1604, and LOC 1613 may indicate high accelerometry even thou user 800 is moving slowly. Accordingly, in the bicycle case, the user 800 although moving slowly is not at rest and TRHT may not be accurately determined. As another example, if user 800 is at home in a relaxing environment and is working to solve a complex technical problem, accelerometry may be low, motion signals may be low, and yet arousal related signals may be high due to heightened mental activity needed to solve the complex technical problem. Accordingly, arousal at stage 1456 may be high as the user 800 is stressed (e.g., not necessarily in a bad way) by the problem solving in a way that affects mind 800m and other physiological parameters of the user 800's body that may manifest as arousal and/or HR, HRV, RES, etc. Therefore, the user 800 may be at rest and not in motion, but rather is stressed and TRHS may not accurately be determined.

Upon determining TRHR (e.g., in bpm), the data for TRHR may be used to compare with one or more other biometric indicators, arousal indicators, I/C/N indicators, fatigue indicators, or others from sensor system 340 and/or from datum's in FIG. 13, for many purposes including but not limited to coaching the user 800, notifications, and reports, just to name a few. As one example, device 100 may notify user 800 that a quality of the user's sleep was not good this Saturday morning using TRHR and an indication of inflammation by device(s) 100. A sleep history (e.g., 1342 in FIG. 13) of the user 800 may indicate that indications of inflammation have occurred in past Saturday mornings and were not present in the user 800 on Friday's the day before. Coaching of user 800 may comprise alerting the user 800 to activities on Friday (e.g., in the evening after work) that may be causes of the inflammation and a suggested remedy for the inflammation (e.g., drink less alcohol on Friday nights).

As another example, if the user 800 historically has a HR (e.g., HR 1383 in FIG. 13) after working out of X bpm and the difference between that HR and the TRHR is a delta of Δ=5 bpm, and recently after working out a delta between the user's HR and TRHR is Δ=12 bpm, then the 7 bpm difference between the users current workout regime and the users historical work regime may be an indication of overtraining by the user 800. Moreover, I/N/C indicators and/or SNS indicators may confirm that the overtraining has resulted in inflammation, dehydration if the user 800 did not properly hydrate during his/her workout, and increased arousal in the SNS of user 800 due to physical stress and/or injury caused by the overtraining. The overtraining may result in user 800 becoming fatigued, in which case GAIT DETC 1630 may determine the user 800 is slower after the workout because the overtraining may have led to injury or affected user 800's state of mind 800m (e.g., as measured by arousal). IMG DETC 1631 may process image data (e.g., from 369) to detect facial expressions, micro-expression, body posture, or other forms of image data that may be used to determine mental and/or physical state of user 800, such as injury and/or fatigue from over training, fatigue caused by other factors, lack of sleep or poor sleep, inflammation (I), contraction (C), just to name a few. Device 100 may notify the user 800 of the overtraining and its indicators (e.g., increased HR, indications of inflammation (I), contraction (C), etc.) and coach the user 800 to drink more fluids to reverse the dehydrations, do fewer repetitions as determined by historical exercise data (e.g., 1338 of FIG. 13) or to rest for 20 minutes after a hard workout, for example. The foregoing are non-limiting examples of how passive determinations of TRHR (e.g., 24/7 and over extended periods of time) may be used and other scenarios may be possible. Moreover, each determination of TRHR may be accomplished without any action on part of the user 800 and without the user 800 even having knowledge that device 100 is currently parsing relevant sensors, analyzing sensor signals, etc. as part continuing process of passively measuring TRHR. As one example, the user 800 may sit down in chair in a hotel lobby to rest/relax for 15 minutes. During that 15 minutes the user 800 is not asleep, is not stressed, and is still (e.g., low accelerometry). Device(s) 100 may have parsed the relevant sensors and determined a TRHR for the user 800 without the user 800 commanding that action or even being aware of it having occurred. The TRHR that was determined in the 15 minutes may be stored as historical data and/or may replace and/or update a prior TRHR measurement.

Referring back to FIGS. 8A-8G, non-limiting examples of when TRHR may be determined by device(s) 100 include but are not limited to: in FIGS. 8B, 8C and 8F, the user 800 is not at rest, is in motion, has accelerometry not consistent with being at rest and awake, therefore TRHR may not be determined; in FIG. 8G where if user 800 is asleep, then user 800 is not awake even thou accelerometry may be consistent with little or no motion, therefore TRHR may not be determined; in FIG. 8G where if user 800 is awake and resting by lying down, then accelerometry may be consistent with little or no motion and if there are no arousal issues in the SNS, then TRHR may be determined; in FIG. 8E where if user 800 is awake and resting by sitting down, then accelerometry may be consistent with little or no motion, and if there are no arousal issues in the SNS, then TRHR may be determined; and in FIG. 8D where if user 800 is awake, and standing, then accelerometry may or may not be consistent with little or no motion, and there may be arousal issues in the SNS, then TRHR may not be determined as standing may not be considered to be a state of resting because some physical activity is required for standing. However, the scenario of FIG. 8D may also be a corner case where user 800 may be at rest, have low or no accelerometry, and have no arousal issues in the SNS such that this corner case may in some examples allow for a determination of TRHR. As to FIG. 8E, if user 800 is sitting at rest in a moving object such as a car, train, plane, etc., then low accelerometry, and no arousal issues from the SNS may still allow for a determination of TRHR and data from LOC/GPS 337 may be analyzed to determine that some accelerometry or other motion may be attributed to the vehicle the user 800 is sitting in.

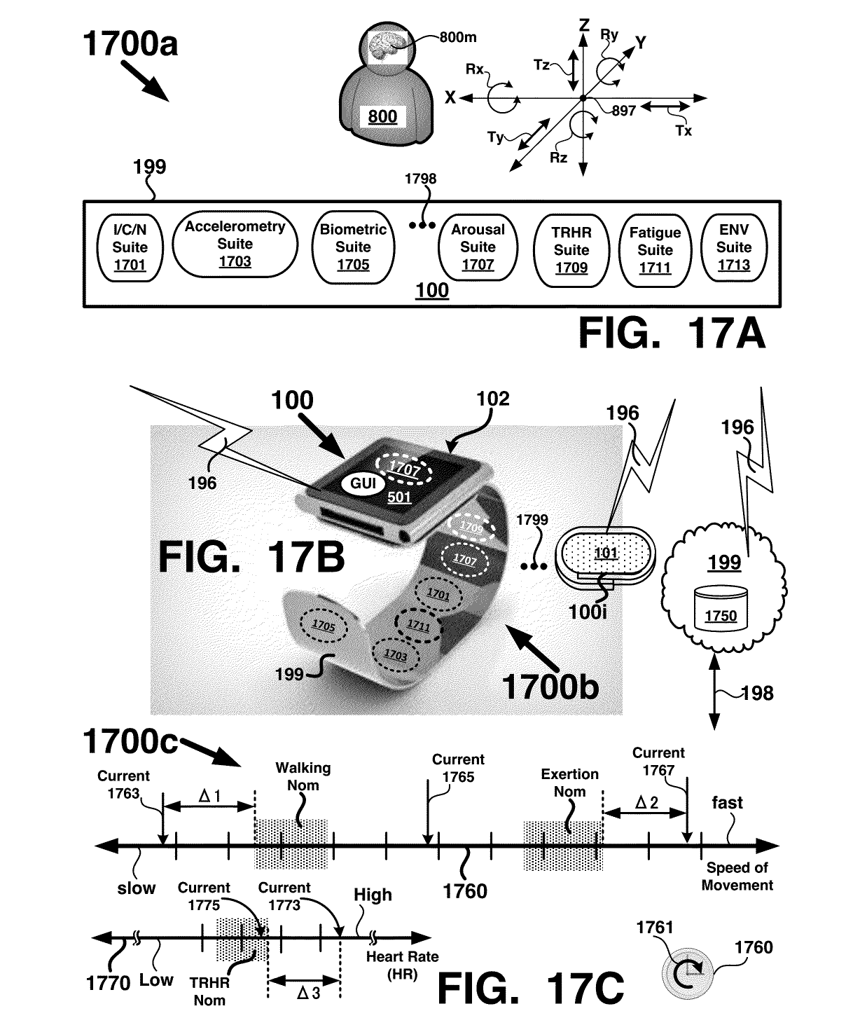

Attention is now directed to FIG. 17A where a block diagram of one example 1700a of sensor platform in a wearable device 100 to passively detect fatigue of a user (e.g., in real-time) that includes a suite of sensors including but not limited to sensor suites 1701-1713. Devices 100 may include all or a subset of the sensor suites 1701-1713. Sensor suites 1701-1713 may comprise a plurality of sensors in sensor system 340 that may be tasked and/or configured to perform a variety of sensor functions for one or more of the suites 1701-1713. For example, biometric suit 1705 may use one or more of the same sensors as the arousal suite 1701, such as a GSR sensor. As another example, accelerometry suit 1703 may use one or more motion sensors that are also used by the fatigue suite 1711. As yet another example, I/C/N suite 1701 may use sensors that are also used by the arousal 1707, biometric 1705, and TRHR 1709 suites. Accelerometry suite 1703 may use one or more motion sensors (e.g., accelerometers, gyroscopes) to sense motion of user 800 as translation and/or rotation about X-Y-Z axes 897 as described above. Sensor suites 1701-1713 may comprise one or more of the sensor devices (e.g., 1601-1617, GAIT 1381) described above in reference sensor system 340 in FIG. 16. Sensor suites 1701-1713 may comprise a high-level abstraction of a plurality different types of sensors in device 100 that may have their signals processed in such a way as to perform the function of the name of the suite, such as a portion of the plurality different types of sensors having their respective signals selected for analysis etc. to perform the I/C/N function of determining whether or not user 800 is in an inflammation state, a nominal state or a contracted state, for example. Therefore, a sensor suite may not have dedicated sensors and may combine sensor outputs from one or more of the plurality of different types of sensors in device 100, for example.

In FIG. 17B, one example 1700a of a wearable device 100 to passively detect fatigue of a user 800 is depicted having a chassis 199 that includes a plurality of sensor suites 1701-1711 positioned at predetermined locations within chassis 199. For example, sensors for detecting biometric signals related to arousal of the SNS for arousal suite 1707 may be positioned at two different locations on chassis 199, and those sensors may be shared with other suites such as biometric suite 1705. There may be more or fewer devices 100, 100i than depicted as denoted by 1799. Device 100i may have different sensor suites than device 100, such as accelerometry suite 1703, biometric suite 1705, and ENV suite 1713; whereas, device 100 may have all of the suites 1701-1713, for example. Device 100 and its suites (e.g., arousal 1701, biometric, accelerometry 1703, and fatigue 1713) may be used for passively determining fatigue in user 800, and may also use data from sensor suites in device 100i (e.g., accelerometry suite 1703 in 100i) to aid in its determination of fatigue. Data including sensor signal data may be shared between devices 100 and 100i via wireless communication link 196, for example. Data from one or more sensor suites may be wirelessly communicated to an external system such as 199 or 999, for example. Data from any of the sensor suites 1701-1713 in any of the devices (100, 100i) may be internally stored (e.g., in DS 320), externally stored (e.g., in 1750) or both. Data may be accessed internally or externally for analysis and/o for comparison to norms (e.g., historically normal values) for the user 800, such as comparing a current HR of user 800 to historical data for a previously determined TRHR of user 800.

In FIG. 17C one example 1700c of speed of movement and heart rate (HR) as indicators of fatigue captured by sensors (e.g., one or more sensor suites of FIGS. 17A-17B) in communication with a wearable device 100 to passively detect fatigue of a user 800 are depicted. Here, sensors used for detecting speed of movement and HR may reside on the device 100, may reside in another device 100 or both. Speed of movement 1760 of user 800 may range from slow (e.g., dragging of feet) to fast (e.g., walking briskly, jogging, or running). HR 1770 may range from low to high (e.g., in bpm). For purposes of explanation only, assume device 100 has sensor suites: 1703 for accelerometry; 1705 for biometrics; and 1711 for fatigue. The accelerometry suite 1703 may include the aforementioned motion sensors (e.g., gyroscope, multi-axis accelerometer), and may also access location data and/or GPS data (e.g., 1613, 1360) to determine distance travelled, speed by dividing distance traveled by time, or to determine if user 800 is more or less remaining in the same location (e.g., a room). Biometric suite 1705 may include sensors for detecting HR, HRV, respiration (RESP), GSR, EMG or others; however, biometric suite 1705 may also access historical or nominal (e.g., normal) data that may be used for comparing current sensor data with normal data for user 800. Device 100 may operate to passively determine fatigue in user 800 on a continuous basis (e.g., 24/7) as denoted by clock 1760 and interval 1761 which cycles continuously in 24/7 mode or less if the mode is intermittent (e.g., every two hours).

Now as for speed of movement 1760, three examples of how accelerometry sensor data and optionally other data such as location data, time of day, day of the week, and historical/normal values for user 800 may be used to determine whether or not the user 800 is fatigued will be described. In a first example, user 800's speed of movement is slow 1763 based on accelerometry data and location data being processed to determine that user 800 is moving slowly at 11:00 am on a Wednesday (e.g., at a time the user 800 is usually walking briskly between college classes). Historical data for the time of day and day of the week (11:00 am and Wednesday) include a range of normal walking speeds for user 800 denoted as “Walking Nom”. Device 100 and/or an external system may process the sensor data, nominal historical data, and optionally other data (e.g., biometric data) to determine that a calculated difference between the current speed of 1763 and the historical norms, denoted as Δ1 may be large enough to indicate fatigue in user 800. As another example, if during strenuous physical activity (e.g., athletic training) historically normal values for speed of movement are denoted by “Exertion Nom” and current sensor data indicates speed of movement is fast at 1767, a calculated difference between the current speed of movement 1767 and the historical norms, denoted as Δ2 may be large enough to indicate fatigue in user 800. In the first example, the indicated fatigue that is causing user 800 to move slower than normal may be due to any number of causes, but as an example, the cause may be mental stress due to studying and may also be due to lack of sleep from staying up late to get the studying done. One or more items of data described above in reference to FIG. 13 may be accessed to determine causation and to provide coaching, avoidance, notifications, reports, etc. For example, the accelerometry suite 1703 may be used to determine length of sleep by analyzing a time difference between motion signals indicating the user 800 has gone to sleep (low accelerometry) and later indicating the user 800 has awaken (higher accelerometry). That time difference may indicate the user 800 got three hours of sleep instead of a normal six hours. Coaching may include recommending getting at least two more hours of sleep, not drinking caffeine right after getting up, and not skipping breakfast. Location data and data on eateries may be used (e.g., see FIG. 13) to determine that the user 800 has not visited the normal locations for breakfast prior to experiencing the slower movement and may be skipping breakfast due to lack of time to eat. Avoidance may include temporal data having information on dates for exams and instructing the user 800 to sleep at least five hours and eat breakfast several days before exams begin to prevent the user 800 from falling into the prior pattern of inadequate sleep and nutrition during exams.

In the second example, Δ2 may indicate overtraining on part of the user 800 that may affect other body functions, such as HR, HRV, inflammation, etc. As one example, current speed of movement 1767 may have strained a muscle in user 800's thigh and lead to systemic inflammation (e.g., the I in I/C/N) and that inflammation has elevated the user 800's HR to a current high value of 1773 such that there is a difference between current HR 1773 and the user 800's TRHR of “TRHR nom”. The normal value for TRHR may be determined as described above and may be stored for later use by devices 100 (e.g., see FIG. 13). Device 100 and/or an external system (e.g., 999) may determine that Δ2 in combination with Δ3 are indicative of fatigue in user 800. Coaching may include recommending user 800 abstain from athletic activities, get rested, and address the indicated inflammation (e.g., strain to thigh muscles). Avoidance may include recommending the user take water breaks and/or rest breaks during the athletic activities as opposed to non-stop exertion from the beginning of the activity to the end.

The examples depicted are non-limiting and data for normal values or ranges of normal values may be stored for later access by devices 100 and/or external systems to aid in determining fatigue, I/C/N, true resting heart rate, stress, etc. As another example, current speed of movement 1765 when analyzed may not trigger any indication of fatigue as its associated accelerometry is not slow or fast, but somewhere in between, or some other metric such as current HR 1775 being within a normal range for TRHR. Current speed of movement 1765 may be associated with low accelerometry but with a speed that is faster than Walking Nom”, and may be an indication that user 800 is riding on public transit and may be sitting down thus giving rise to a HR that is within the normal for TRHR, such that the data taken as a whole does not indicate fatigue.

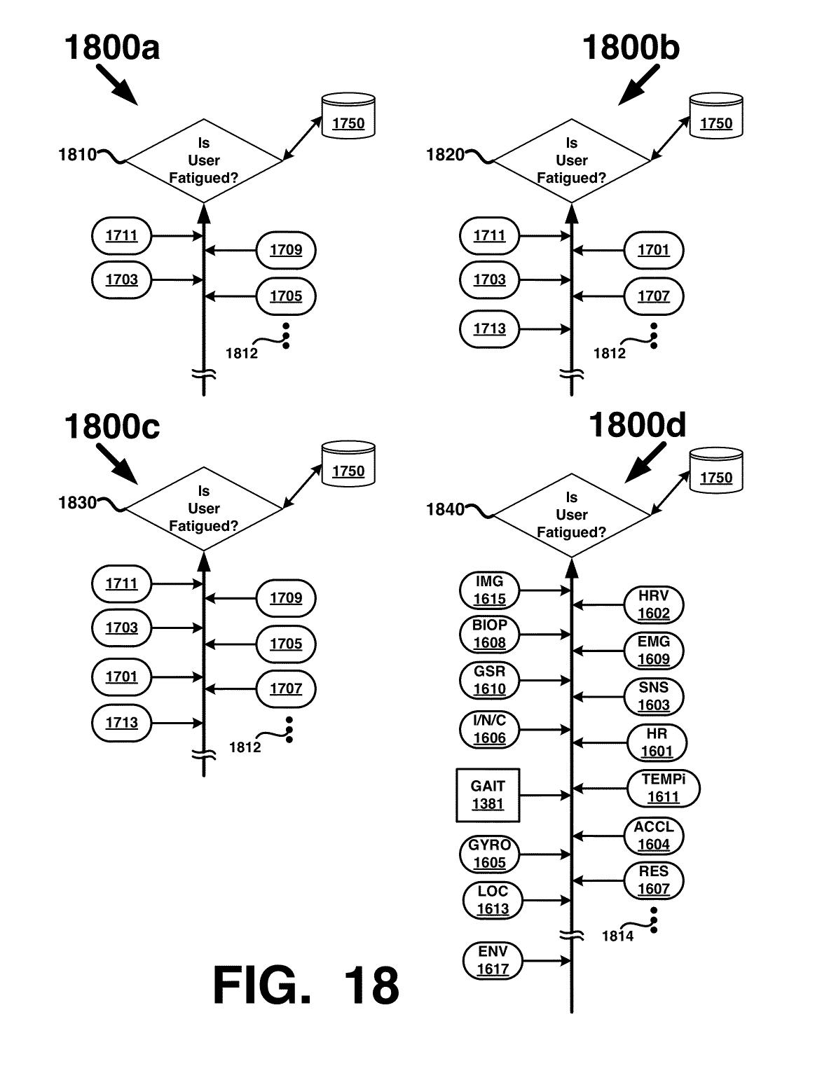

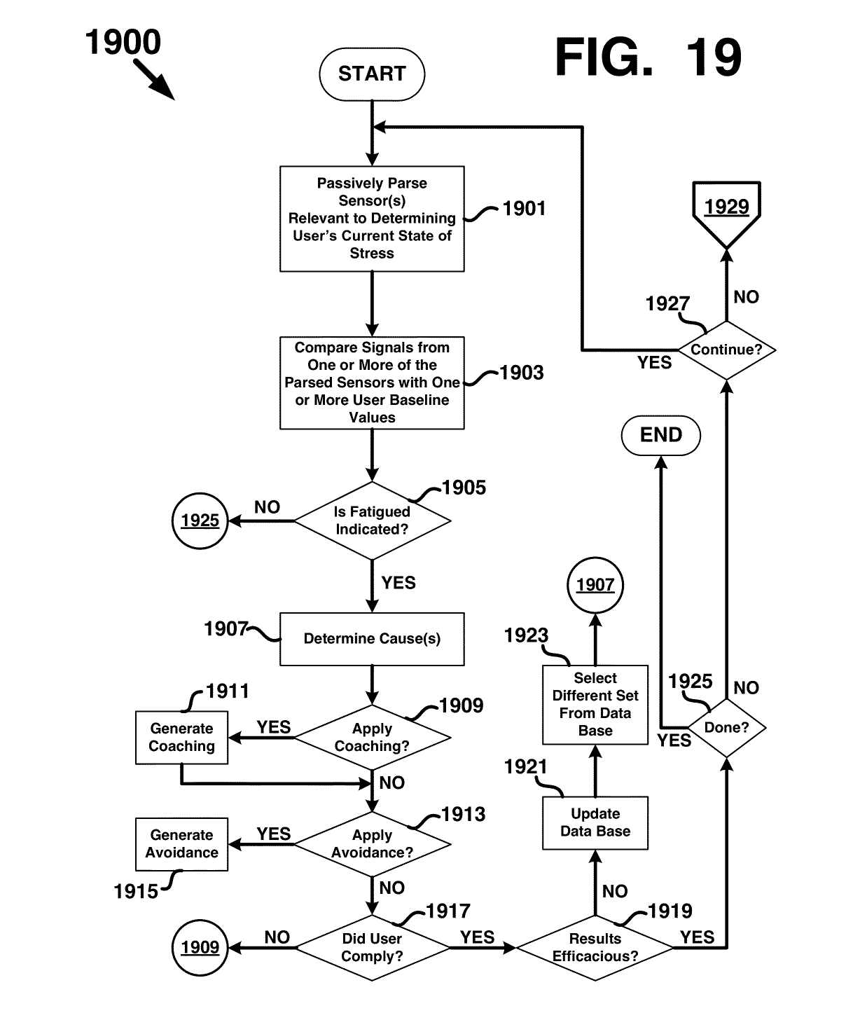

Referring now to FIG. 18 where examples 1800a-1800d of sensor inputs and/or data that may be sourced internally or externally in a wearable device 100 to passively detect fatigue of a user are depicted. Stages depicted in examples 1800a-1800d may be one of a plurality of stages in a process for passively determining fatigue (e.g., in real-time). Data, datum's, items of data, etc. depicted in FIGS. 13 and 16 may be used for in examples 1800a-1800d.

In example, 1800a, a stage 1810 for passively determining fatigue in a user 800 may comprise data from one or more sensor suites: accelerometry 1703; biometrics 1705; TRHR 1709; fatigue 1711; and more or fewer suites as denoted by 1812. Moreover, data 1750 may be accessed (e.g., wirelessly for read and/or write) by one or more devices 100 to make the determination at stage 1810.

In example 1800b, a stage 1820 for passively determining fatigue in a user 800 may comprise data from one or more sensor suites: I/C/N 1701; accelerometry 1703; arousal 1707; fatigue 1711; ENV 1713; and more or fewer suites as denoted by 1812. Furthermore, data 1750 may be accessed.

In example, 1800c, a stage 1830 for passively determining fatigue in a user 800 may comprise data from one or more sensor suites: I/C/N 1701; accelerometry 1703; biometrics 1705; arousal 1707; TRHR 1709; fatigue 1711; ENV 1713; and more or fewer suites as denoted by 1812. Furthermore, data 1750 may be accessed.

In example 1800d, a stage 1840 for passively determining fatigue in a user 800 may comprise data from one or more sensors: IMG 1615; BIOP 1608; GSR 1610; I/N/C 1606; GAIT 1381; GYRO 1605; LOC 1613; ENV 1617; HRV 1602; EMG 1609; SNS 1603; HR 1601; TEMPi 1611; ACCL 1604; and RES 1607, and more or fewer sensors as denoted by 1814. Furthermore, data 1750 may be accessed. Data 1750 may include one or more of the items of data depicted in FIG. 13. Sensors and/or sensor suites in examples 1800a-1800d may be accessed, parsed, read, or otherwise in real-time and optionally on a 24/7 basis, for example.

Turning now to FIG. 19 where one example of a flow diagram 1900 for passively detecting fatigue in a user 800 is depicted. Flow 1900 may be executed in hardware, software or both and the hardware and/or software may be included in one or more of the devices 100 and/or in one or more external devices or systems (e.g., 199, 960, 999). At a stage 1901 sensor relevant to determining a current state of stress (or lack of stress) may be parsed (e.g., have their signal outputs read, sensed, by circuitry in device 100) passively, that is without intervention on part of user 800. At a stage 1903 signals from one or more of the relevant sensors that were parsed may be compared with one or more baseline (e.g., normal or nominal) values (e.g., baseline data) as described above (e.g., in FIG. 17C). The baseline values/data may be from an internal data source, an external data source or both as described above. The comparing may be accomplished in hardware (e.g., circuitry), software or both. The hardware and/or software for the stage 1903 and other stages of flow 1900 may reside internal to one or more devices 100, external to one or more of the devices 100 or both. At a stage 1905 a determination may be made as to whether the comparison at stage 1903 is indicative of fatigue (e.g., chronic stress) in user 800. If a NO branch is taken, then flow 1900 may transition to another stage, such as a stage 1921, for example. If a YES branch is taken, then flow 1900 may transition to a stage 1907. At the stage 1907 one or more causes for the indicated fatigue may be determined using one or more items of data and/or sensor signals described herein, such as describe above in reference to FIGS. 9-11 and 13-18, for example.

At a stage 1909 a decision may be made as to whether or not the determined cause(s) may require applying coaching. If a YES branch is taken, then flow 1900 may transition to a stage 1911 were coaching data (e.g., ASCII text, HTML, XML, SMS, email, digital audio file, or other format of data) may be communicated to user 800, a client device (e.g., 999), one or more devices 100 (e.g., see 501 in FIG. 5) or external device or system. Flow 1400 may transition from stage 1911 to a stage 1913 as will be described below, so that application of avoidance may be decided based on the determined cause(s) at the stage 1907. If a NO branch is taken, then flow 1900 may transition to the stage 1913.

At the stage 1913 a decision may be made as to whether or not the determined cause(s) may require applying avoidance. If a YES branch is taken, then flow 1900 may transition to a stage 1915 were avoidance data (e.g., ASCII text, HTML, XML, SMS, email, digital audio file, or other format of data) may be communicated to user 800, a client device (e.g., 999), one or more devices 100 (e.g., see 501 in FIG. 5) or external device or system. If a NO branch is taken, flow 1900 may transition to a stage 1917 were a determination may be made as to whether or not the user 800 has complied with the coaching (if generated), the avoidance (if generated) or both. If a NO branch is taken (e.g., compliance of user 800 is not detected), flow 1900 may transition to another stage, such as the stage 1909, where the analysis for coaching and/or avoidance may be repeated. If a YES branch is taken (e.g., compliance of user 800 is detected), then flow 1900 may transition to a stage 1919.

At the stage 1919 a determination may be made as to whether or not the results of user compliance at the stage 1917 have been efficacious, that is, has fatigue (e.g., stress) been reduced or eliminated (e.g., as determined by sensors in device(s) 100, etc.). If a NO branch is taken, then flow 1900 may transition to a stage 1921 where one or more data bases may be updated using data from any of the stages of flow 1900 that may relevant to improving results in future interactions of flow 1900. At a stage 1923, a different set or sets of data may be selected from the data base and flow 1900 may transition to another stage, such as the stage 1907 to re-determine the cause(s) of the fatigue. If a YES branch is taken at the stage 1919, then flow 1900 may transition to a stage 1925 where a determination may be made as to whether or not fatigue detection is completed (e.g., is flow 1900 done?). If a YES branch is taken, then flow 1900 may terminate. If a NO branch is taken, then flow 1900 may transition to a stage 1927 were a determination to continue flow 1900 may be made. If a YES branch is taken, then flow 1900 may transition to another stage, such as the stage 1901, for example. Flow 1900 may continuously execute on a 24/7 basis or in some interval, such as every 10 minutes, for example.

If a NO branch is taken from the stage 1927, then flow 1900 may transition to another flow as denoted by 1929. For example, off-page reference 1929 may represent another flow for determining other activity in body of user 800, such as the flow 1000 of FIG. 10, the flow 1400 of FIG. 14, the flow 1500a and/or 1500b of FIGS. 15A and 15B, for example. As one example, the NO branch from the stage 1927 may transition to flow 1000 for determination of I/C/N and flow 1000 may transition to flow 1400 for determination of TRHR, and then flow 1400 may transition to flow 1900 for determination of fatigue, on so on and so forth. The flows described herein may execute synchronously, asynchronously, or other on one or more devices 100 and execute in sequence, or in parallel.

Although the foregoing examples have been described in some detail for purposes of clarity of understanding, the above-described inventive techniques are not limited to the details provided. There are many alternative ways of implementing the above-described techniques or the present application. Waveform shapes depicted herein are non-limiting examples depicted only for purpose of explanation and actual waveform shapes will be application dependent. The disclosed examples are illustrative and not restrictive.

What is claimed is:

1. A data capable strapband, comprising:
a wearable device including
one or more radios operative for wireless communication using one or more wireless protocols;
a sensor system including
a pressure sensor operative to couple with body tissue and to generate a force signal indicative of pressure applied to the pressure sensor by the body tissue,
a motion sensor operative to generate a motion signal in response to motion of the body tissue,
a plurality of biometric sensors operative to generate biometric signals including arousal signals in response to arousal of the sympathetic nervous system (SNS); and
a processor operative to analyze the force signal, the motion signal, and the arousal signals, the motion and force signals are analyzed to determine motion induced error in the force signal caused by the motion of the body tissue, to subtract out the motion induced error, if present in the force signal, using the motion signal, and to generate a signal indicating an inflammation (I) state, a contraction (C) state, or a nominal (N) state of the body tissue,
the processor operative to communicate information relevant to a sleep state when the motion signal, the arousal signals or both indicate sleep and the I state of the body tissue or the C state of the body tissue are indicated.

2. The data capable strapband of claim 1, wherein the wearable device further includes a display.

3. The data capable strapband of claim 2, wherein the information is presented on the display.

4. The data capable strapband of claim 1, wherein the information is wirelessly communicated to a wireless client device using the one or more radios.

5. The data capable strapband of claim 1, wherein the information comprises one or more types of information selected from the group consisting of a report, coaching, a notification, and avoidance.

6. The data capable strapband of claim 1, wherein the plurality of biometric sensors a plurality of sensors selected from the group consisting of a heart rate sensor, a respiration sensor, a temperature sensor, a skin conductance sensor, a skin conductance response sensor, a galvanic skin response (GSR) sensor, an electromyography (EMG) sensor, an electrodermal activity sensor, and electrodermal response sensor.

7. The data capable strapband of claim 1, wherein the processor is disposed in a system that is external to the wearable device, the force signal, the motion signal, and the arousal signals are wirelessly communicated to the processor using a wireless link between the system and the one or more radios.

8. The data capable strapband of claim 1, wherein the processor accesses external data that is relevant to the sleep state and the external data comprises one or more datum selected from the group consisting of true resting heart rate (TRHR), heart rate variability (HRV), fatigue data, location data, diet data, almanac data, health data, and exercise data.

9. The data capable strapband of claim 1, wherein the processor is included in the wearable device and the processor is in communication with the sensor system.

10. The data capable strapband of claim 1, wherein the information is wirelessly communicated to an external resource.

11. The data capable strapband of claim 10, wherein the external resource comprises one or more resources selected from the group consisting of the Cloud, the Internet, a web page, a web site, a compute engine, data storage, network attached storage (NAS), a server, Flash memory, a hard drive (HD), and a solid state drive (SSD).

12. The data capable strapband of claim 10, wherein the wearable device further includes a location unit in communication with the processor and operative to determine a location of the wearable device when donned, operative to gather location data from an external source or both.

13. The data capable strapband of claim 12, wherein the location unit, the location data or both are global positioning system (GPS) based.

14. The data capable strapband of claim 12, wherein the processor is operative to access the location unit, the location data or both, to processes data from the access, and to include the data from the access in the information when the processing determines that the data from the access is relevant to the sleep state.

15. The data capable strapband of claim 14, wherein when the data from the access is relevant to the sleep state it is included in the information regardless of the I, C, or N states of the body tissue being indicated.

16. The data capable strapband of claim 1, wherein the one or more radios are in wireless communication with one or more other radios in another wearable device over a wireless link, and sensor signals from the sensor system and from another sensor system in the another wearable device are wirelessly communicated over the wireless link and are processed by the processor, by another processor or both to communicate the information relevant to the sleep state.

17. The data capable strapband of claim 16, wherein the another wearable device and the wearable device are donned on different portions of body tissue on a user's body.

18. The data capable strapband of claim 17, wherein the wearable device is donned on an arm body tissue, and the another wearable device is donned on a leg body tissue.

19. The data capable strapband of claim 18, wherein the wearable device and the another wearable device include a flexible chassis, the flexible chassis operative to retain its shape when donned.

20. The data capable strapband of claim 1, wherein the wearable device includes a flexible chassis operative to retain its shape when donned on the body tissue.
